(19) United States
(12) Patent Application Publication
(10) Pub. No.: US 2026/0337542 A1
(43) Pub. Date: Oct. 29, 2026

(54) APPARATUS, SYSTEMS AND METHODS FOR AUTOMATED BIOPROCESSING

(71) Applicant: CELLUAR ORIGINS LIMITED, Melbourn (GB)

(72) Inventors: Dan Strange, Melbourn (GB); Simon Lyons, Melbourn (GB); Zak Karimjee, Melbourn (GB); Maria Carnarius, Melbourn (GB); Rob Selby, Melbourn (GB); Nathan Wilkinson, Melbourn (GB); Duncan Young, Melbourn (GB); Alex Coney, Melbourn (GB); Chris Hemingway, Melbourn (GB)

(21) Appl. No.: 19/107,366

(22) PCT Filed: Aug. 31, 2023
(86) PCT No.: PCT/GB2023/052258

§ 371 Date: Feb. 27, 2025

(30) Foreign Application Priority Data
Aug. 31, 2022 (GB) 2212678.3
Aug. 31, 2022 (GB) 2212680.9
Aug. 31, 2022 (GB) 2212681.7
Aug. 31, 2022 (GB) 2212682.5

(51) Int. Cl. C12M 1/36; C12M 1/00; C12M 3/00

(52) U.S. Cl. CPC C12M 41/48; C12M 23/48; C12M 23/52

(57) ABSTRACT

A bioprocessing system, comprising: a plurality of shelving frames, each shelving frame configured to hold a plurality of cell culture chambers; and an automated system configured to: move a cell culture chamber around the bioprocessing system; and manipulate a fluid connection between a cell culture chamber and a bioprocessing consumable; wherein at least one of the shelving frames is movable relative to at least one other of the shelving frames whereby to facilitate or inhibit access to a cell culture chamber held on one of the shelving frames.

### FIELD OF THE INVENTION

The present disclosure relates to bioprocessing, and more specifically to apparatus, systems and methods for automated bioprocessing.

### BACKGROUND

Therapeutics are increasingly using cells rather than small molecules as the starting point. The approaches to manufacturing these products are rapidly evolving to keep up with constantly emerging new therapies. In recent years, there has been an increased use of a number of new classes of cell therapies. One class is autologous cell therapies.

Autologous cell therapies are a promising class of therapy, which have significant clinical and commercial potential ranging from treating cancer to fixing genetic defects. These therapies involve taking cells from a patient, manipulating the cells over the course of days to weeks, and re-introducing the cells back into that patient's body to produce a therapeutic effect. The steps taken during autologous cell therapies are often complex; for example, a typical CAR-T process may involve a sequence of steps starting with a cryopreserved leukopak, thawing, washing to remove DMSO, enrichment of T cells, activation, transduction, expansion, concentration, formulation fill finish into an IV bag, and cryopreservation, with several other intermediate washing steps.

Bioprocessing systems have been developed for carrying out the above steps. Within such a bioprocessing system, various different consumables (which may also be referred to as “containers”, “chambers” or “vessels”) may be required for holding media, reagents and/or cells, and fluid may be transferred between the consumables throughout the cell therapy process. The term “consumable” is preferably used to refer to a “single-use” element or component of the system.

Some of the processes discussed above such as activation, transduction and expansion may be carried out within a cell culture chamber, which may be incubated, such as in a bioreactor. During one or more of those steps, various functions need to be provided by the bioreactor, such as perfusion, gas and nutrient transport, reagent mixing, media conditioning and heat exchange. However, existing bioreactors, consumables and bioprocessing systems have a number of problems.

Many patient samples may be processed simultaneously using the bioprocessing system in individual cell culture chambers. In particular, many patient samples may need to be stored and incubated simultaneously; in an exemplary 7-day bioprocessing operation, the cell culture chambers may spend about 90% of the time in a bioreactor and/or an incubator of some kind. This may present a number of challenges.

Firstly, there is a need for a large amount of storage or incubation space if several bioprocessing operations are to be performed simultaneously. Secondly, the cell culture chambers need to be accessible when being stored or incubated to facilitate manipulation and connection to other parts of the bioprocessing system, such as at processing stations. Thirdly, in order to prevent contamination of the cell culture chambers, the sterility requirements for bioprocessing are very high, and thus the bioprocessing system needs to be kept very clean. Fourthly, the cell culture chambers may need to be stored in precisely controlled conditions, such as at a particular temperature for incubation.

As a result of the above challenges, existing bioprocessing systems may take up a significant amount of space, which increases the construction and operating costs for the system; for example, by having a larger volume of air it is more expensive to keep the air sterile by filtering, and also requires more energy to control the temperature of the system. In addition, a larger area increases the cleaning burden when cleaning and disinfecting the surfaces within the bioprocessing system, and it may be difficult to access all parts of the bioprocessing system in order to perform such cleaning.

Therefore, it is an object of the present invention to address the problems discussed above. It is also desirable to provide an improved cell culture chamber and bioreactor.

### SUMMARY OF INVENTION

Described herein is a bioprocessing system, comprising: a plurality of shelving frames, each shelving frame configured to hold a plurality of bioreactor apparatus, each bioreactor apparatus being configured to receive at least one cell culture chamber; and an automated system configured to: move a cell culture chamber around the bioprocessing system; and manipulate a fluid connection between a cell culture chamber and a bioreactor apparatus; wherein at least one of the shelving frames is movable relative to at least one other of the shelving frames whereby to facilitate or inhibit access to a bioreactor apparatus held on one of the shelving frames.

Also described herein is a bioprocessing system, comprising: a plurality of shelving frames, each shelving frame configured to hold a plurality of cell culture chambers (e.g., cell culture chambers received in a plurality of bioreactor apparatus held on each shelving frame); and an automated system configured to: move a cell culture chamber around the bioprocessing system; and manipulate a fluid connection between a cell culture chamber and a bioprocessing consumable (e.g., a bioreactor apparatus); wherein at least one of the shelving frames is movable relative to at least one other of the shelving frames whereby to facilitate or inhibit access to a cell culture chamber (e.g., when received in a bioreactor apparatus) held on one of the shelving frames.

Each shelving frame may be configured to hold a plurality of bioreactor apparatus, each bioreactor apparatus being configured to receive at least one cell culture chamber. The bioprocessing consumable may be provided by a bioreactor apparatus held on one of the shelving frames.

Alternatively, the automated system may be configured to manipulate a fluid connection between the cell culture chamber and the bioprocessing consumable at a bioprocessing apparatus that is located in the bioprocessing system separately to the plurality of shelving frames. The bioprocessing apparatus may be a processing station, such as a processing station for supplying or removing fluid to a connected cell culture chamber. The automated system may move the cell culture chamber from its shelving frame to the bioprocessing apparatus, manipulate the fluid connection to the bioprocessing consumable, and (following a transfer of fluid between the cell culture chamber and the bioprocessing consumable) may disconnect the fluid connection and return the cell culture chamber to the shelving frame. The bioprocessing consumable may be a container of reagent or media, may be an empty consumable for receiving a waste product from the cell culture chamber, or may be an output consumable for receiving cultured cells.

The bioreactor apparatus may be the bioreactor apparatus described below and herein or may be a separate bioreactor apparatus. The bioreactor apparatus may be configured to incubate the cell culture chamber.

By having one or more shelving frames that are movable relative to one or more other shelving units, a number of advantages are provided.

Firstly, the overall space of the environment can be reduced. When access is not required to one of the shelving frames, the shelving frames can be moved closer to each other to reduce their separation, and only moved apart when access is required (such as to allow for moving, manipulation and/or connection of a cell culture chamber on the shelving frame). Not only does this more efficiently utilise the physical space, it also reduces the running costs for the bioprocessing system. For example, where the bioprocessing system is provided in an enclosed space with environmental control (e.g., including an HVAC system), the movable shelves allow the total volume of the enclosed space to be reduced, which reduces the operating costs for the environmental control. In addition, a small volume means that there is less air to be heated if used as an incubation room (with raised temperature), and less air to be filtered to maintain high sterility requirements (e.g., if used as a class C cleanroom or similar).

Secondly, since the movement of cell culture chambers and manipulation of fluid connections are performed by an automated system, it is particularly important to ensure that the automated system does not misidentify any bioreactor apparatus or other containers, which could result in cross-contamination. By having movable shelves, access may be inhibited to incorrect locations in the bioprocessing system while selectively allowing access to the correct location. This provides an additional check for possible errors in a bioprocessing workflow, thereby reducing the risk of errors by the automated system.

Thirdly, where the bioprocessing system is operated as a cleanroom (e.g., a class C cleanroom), it is particularly important to allow the system to be cleaned and/or disinfected efficiently. By allowing movement of the shelving frames, the entire floor space and/or wall space of the bioprocessing system may be accessed, thereby allowing for more thorough cleaning of the bioprocessing system.

Fourthly, by having shelving frames that are movable to inhibit access to particular shelving frames at certain times, the surfaces of the shelving frames that hold the bioreactors are not exposed to the surroundings and are therefore less likely to receive dust deposits. This means that the movable shelving frames further decreases the cleaning burden of the bioprocessing system, since dust (or other contaminants) cannot settle as easily on or around the bioreactors.

Preferably the automated system comprises a tube welder in order to manipulate the fluid connection, such as a tube welder on a robotic device. Alternatively, the fluidic connection may be manipulated using any other type of reusable or reversible aseptic connector. The skilled person having the benefit of this disclosure will be aware of connectors suitable for this purpose. Alternatively, the fluid connection may be manipulated within a locally aseptic environment such as a sterilisation chamber, which may use an autoclave, laser, steam disinfection or a sterilant. By using an automated system for moving and manipulation of the cell culture chambers (rather than a human operator), the bioprocessing system is less likely to be contaminated by human operators during routine use (who are the largest source of dust), which further decreases the cleaning burden.

The shelving frames may be mounted on a track system along which each frame can be moved within the enclosed space.

The automated system may comprise: a gantry comprising a horizontal member mounted slidably to at least one vertical member and configured such that the horizontal member can be moved along the vertical member; and at least one robotic device mounted slidably to the horizontal member and configured such that the robotic device can move along the horizontal member.

Alternatively or additionally, the automated system may comprise a robotic device configured to move across a floor of the bioprocessing system whereby to access each of the cell culture chambers (e.g., when received in a bioreactor apparatus).

The (or a) robotic device of the automated system may be configured to releasably engage with a cell culture chamber whereby to move said cell culture chamber. The (or a) robotic device of the automated system may be configured to manipulate said fluid connection. A single robotic device may be configured both to move a cell culture chamber and to manipulate said fluid connection.

The plurality of shelving frames may be disposed within an enclosed space having an opening through which a cell culture chamber can be passed, the opening comprising a pair of doors defining a compartment therebetween into which the cell culture chamber can fit with both doors closed.

The enclosed space may be substantially thermally sealed thereby to inhibit the transfer of heat between the enclosed space and its surroundings.

At least one bioreactor apparatus and/or at least one cell culture chamber in each shelving frame may have an individual machine-readable identifier, for example a QR code.

Also described herein is a cell culture chamber for use with a bioreactor apparatus within a bioprocessing system, comprising: a first housing having an upper surface and a lower surface, with one or more open fluidic channels formed on at least one of said upper and lower surfaces; at least one sealing layer arranged to be affixed onto at least one of the upper or lower surface of the first housing, the sealing layer arranged to seal said one or more open fluidic channels formed on a corresponding surface of the first housing, thereby forming closed fluidic paths through which fluid can be routed; a second housing configured to contain a volume of fluid, the second housing being joined to the first housing; and one or more fluid ports provided on at least one of the first housing and the second housing, the one or more fluids ports being fluidly connected to said one or more fluidic channels.

By providing a first housing and a sealing layer to form closed fluidic paths, all fluid paths are integrated on one surface, thereby simplifying the manufacture of the chamber. The sealing layer may be a film that may be flexible, or it may be a less flexible (e.g. rigid) member, such as a (e.g. plastic) plate. Preferably, the second housing is arranged to contain a volume of fluid beneath the lower surface of the first housing, in use. Alternatively, the second housing may contain a volume of fluid above the upper surface of the first housing.

The sealing layer may be a first sealing layer affixed on the upper surface of the first housing, and the chamber may further comprise a second sealing layer affixed on the lower surface of the first housing, such that the first housing is sandwiched between the first sealing layer and the second sealing layer. By providing two sealing layers the moulding of the first housing may be simplified and/or it may be easier to have more functionality embedded into the cell culture chamber. One or both of the sealing layers are preferably laminated or heat sealed onto the first housing. Alternatively, the sealing layers may be welded, solvent bonded, or diffusion bonded onto the first housing.

The second housing may comprise a gas permeable membrane, preferably located at the bottom of the second housing, preferably comprising silicone. Preferably, a bottom surface of the second housing may comprise a porous structure thereby allowing gas to reach the gas permeable membrane. Alternatively, the gas permeable membrane may define the lower surface of the second housing.

The first housing and second housing may be joined together using an ultrasonic welding technique. The first housing may be welded to the second housing or it may be bonded to the second housing using adhesive, solvent bonding and/or diffusion bonding, for example, to form a fluidic seal. Alternatively, the first and second housing may be clamped together via an O-ring seal.

The cell culture chamber may further comprise a heating element arranged to heat the cell culture chamber. At least a portion of the closed fluidic paths may be arranged to function as a heat exchange loop. In this way, fluid may be gradually heated prior to entry into the second housing. This allows the second housing to be maintained at a more consistent and optimal temperature.

The closed fluidic paths may comprise a sampling loop from which a sample can be extracted. In this way, samples can be extracted from the closed fluidic paths. The sampling loop preferably has a very low dead volume, thereby reducing waste of fluid during a sampling operation.

The sampling loop may be connected to two fluid ports of the cell culture chamber, and the sampling loop may comprise a duct that extends into the second housing. In this way, by increasing pressure in the second housing (such as by shutting off the waste outlet port and continuing to pump fluid into the second housing), a sample of fluid may be forced into the sampling loop via the duct. If one of the two fluid ports connected to the sampling loop is also shut, a continuous stream of sampled fluid may be produced within the sampling loop. Subsequently, a pressure difference may be applied between the two fluid ports connected to the sampling loop, thereby transporting the sample of fluid out one of the fluid ports such as for external inspection. Advantageously, this may allow a specific and controllable volume of fluid to be sampled from the cell culture chamber.

The cell culture chamber may further comprise a mixer for reagent or gas exchange.

The cell culture chamber may further comprise means for inspecting the fluid.

The cell culture chamber may further comprise means for pumping fluid through the closed fluidic paths. In this way the cell culture chamber can be operated in perfusion. The means for pumping may comprise a portion of flexible tube adapted to be engaged by a peristaltic pump. Alternatively, the cell culture chamber may comprise an integrated peristaltic chip configured to be acted on by an external driver. In other words, the cell culture chamber may at least partially comprise a peristaltic pump which may be externally driven.

The cell culture chamber may further comprise at least one of means for filtering; means for washing the cells; and means for measuring cell count.

The cell culture chamber may further comprise at least one electrical sensor configured to measure impedance and/or capacitance of a fluid within the cell culture chamber. The electrical sensor may be embedded in the gas permeable membrane. The electrical sensor may comprise a plurality of electrodes.

The cell culture chamber may further comprise a pH indicator arranged such that the pH of a fluid contained within the second housing can be determined via inspection of the pH indicator. For example, the pH indicator may be a pH label located on the second housing.

The cell culture chamber may have a planar configuration that is substantially flat such that it can be inserted into an elongate slot of a receptacle. The ratio between the length of the first and/or second housing to the height of the first and/or second housing may be at least 1:1, and more preferably at least 4:1. As used herein, the length of the second housing refers to the length measured along a perfusion direction, which may be a direction in which media flows through the second housing.

The cell culture chamber may further comprise an engagement feature arranged to be engaged by an automated mechanism whereby to facilitate manipulation of the cell culture chamber by the automated mechanism. The engagement feature may be provided by at least one protrusion arranged on an external surface of the cell culture chamber. Said external surface is preferably a surface that remains accessible by the automated mechanism when the cell culture chamber is inserted into a bioreactor apparatus, such as a front surface of the cell culture chamber. In this way, the engagement feature on the front surface of the cell culture chamber may be engaged by the automated mechanism to insert and remove it from a (front-facing) opening to a cavity of a bioreactor apparatus. The engagement feature may be provided by a portion of the first housing extending beyond the second housing, e.g. to form a protruding portion or element that can be engaged/gripped by an automated mechanism.

The one or more fluid ports may be arranged in a linear configuration, preferably on a common surface of the cell culture chamber. The one or more fluid ports may each be configured to form a fluidic seal with a fluid conduit received therein. A minimum width of the fluidic channels may be less than 3 mm, and preferably less than 1 mm. In this way, it is possible to provide low dead volumes. In contrast, in systems that use standardised tubing (to interface with sterile tube welding systems), it is much more difficult to provide low dead volume.

Also described herein is a fluid manifold for supplying fluid to a bioprocessing apparatus, comprising: a housing; a plurality of flexible fluid conduits extending through the housing, each fluid conduit being configured to be fluidly coupled to a corresponding port provided on the bioprocessing apparatus; and a plurality of controllable valves, each valve being configured to facilitate control of the flow of fluid through at least one of said fluid conduits.

The plurality of fluid conduits may be configured as a matrix in which at least one further fluid conduit is arranged to provide a fluid connection between two of more of said plurality of fluid conduits, whereby the matrix can be configured to form one or more fluid cross-connections between said fluid conduits. The plurality of conduits may be arranged in a substantially parallel configuration. The at least one further fluid conduit may connect the plurality of conduits in a direction substantially perpendicular to the plurality of parallel conduits.

One or more of said valves may be configured to compress a portion of a corresponding fluid conduit when actuated so as to inhibit the flow of fluid therethrough.

One or more of said valves may be configured to control the flow of fluid through a conduit by way of (e.g., rotation of) a valve element compressing a portion the fluid conduit. For example, the valve element may comprise a pinch mechanism or a rotatable cam.

The one or more valves may be configured to be operable by both human and automated means. For example, the valves may be configured such that they can be operated from different sides of the manifold when fluidly coupled with a bioprocessing apparatus, optionally wherein the valves are configured to be operable by a human on a first (front) side of the manifold and by an automated means on a second (rear) side of the manifold.

One or more of said valves may be operable to be locked into position, preferably by a removable locking member. The locking member is preferably part of a locking mechanism. The locking member may be a locking key. The locking key may have external threading configured to engage with internal threading on a valve aperture corresponding to each valve, whereby rotation of the locking key into the valve aperture compresses and pinches shut a fluid conduit.

One or more of said valves may be disposed within the housing and arranged such that it can be actuated external of the housing. The rear side of the manifold may comprise one or more openings corresponding to each of the one or more valves. In this way, an (automated) valving actuator (such as a solenoid mechanism located on a bioreactor apparatus) may extend into the opening thereby pinching shut a fluid conduit that passes through each valve.

The housing may be mountable to a support, preferably via one or more mounting features provided on the housing, and more preferably provided on one or more sides of the housing.

Also described herein is a manifold for supplying fluid to a bioprocessing apparatus, comprising: a matrix of interconnected fluid conduits arranged to provide a plurality of first openings and second openings, wherein each of the fluid conduits is sealable such that fluid flow can be inhibited between one or more of said first openings and one or more of said second openings. In this way, it is possible to manufacture a single consumable, which can then be easily configured to define various different fluid path configurations, simply by selectively sealing predetermined conduits.

The matrix may consist of the sealable fluid conduits. In other words, the matrix includes only the sealable fluid conduits without the presence of other components. In this way, the matrix is a unitary component formed as a single piece (rather than several conduits that are subsequently attached together), which may be particularly easy to manufacture.

The matrix may be injection moulded. The tube matrix may be manufactured from one common mould tool, thereby simplifying manufacture of the manifold.

The fluid conduits may comprise a thermoplastic. The fluid conduits may be sealed by manipulating the fluid conduit. The sealing may be achieved by heat or RF, for example.

The manifold may be mountable directly to a cell culture chamber by way of a portion of one of more fluid conduits being received into one or more corresponding fluid ports on the cell culture chamber.

The cell culture chamber may be the cell culture chamber described above and herein.

Also described herein is a bioreactor apparatus, comprising: a housing having at least one internal cavity for receiving therein a cell culture chamber, the internal cavity having an opening located on an external front-facing surface of the housing, the housing further configured to have mounted to it at least one holding device for supporting a container of a fluid medium for fluid connection to a cell culture chamber disposed within said internal cavity, said holding device being mountable to the housing via a docking port located in said external front-facing surface of the housing; wherein the housing is configured to incubate the contents of a cell culture chamber disposed within said internal cavity.

In this way, expansion chambers (e.g., cell culture chambers) and associated consumables are both be front-loaded into the bioreactor to facilitate ease of an automated mechanism to perform the loading. Preferably, a container supported by the holding device is elevated relative to said cell culture chamber. In this way, a pump is not required to transfer fluid from the container to the cell culture chamber.

The at least one internal cavity may comprise a push-latch mechanism configured to secure a corresponding cell culture chamber. The push-latch mechanism may be spring-loaded so that the cell culture chamber may be readily removed from the bioreactor apparatus by an automated mechanism.

The housing may be further configured to support a portion of the holding device whereby it rests on the housing.

The bioreactor apparatus may further comprise at least one flow controller arranged on said external front-facing surface of the housing, said flow controller configured to control the flow of fluid medium between a container mounted to the housing and a cell culture chamber disposed within said internal cavity of the housing. In this way, during use, when one or more containers are connected to the cell culture chamber (e.g., with one or more fluid conduits), the flow controllers may inhibit or allow fluid to flow between the containers and the cell culture chamber. For example, the flow controller may be a valving actuator that presses against a fluid conduit (e.g., of a fluid manifold) to control the flow, during use. It will be appreciated that the flow controllers may control the flow of fluid between any container and cell culture chamber during use (since different combinations of cell culture chamber and containers may be used depending upon the specific bioprocessing operation being performed).

The fluid connection between a container of fluid medium and a cell culture chamber may comprise at least one fluid conduit.

The bioreactor apparatus may further comprise at least a device for retaining the fluid conduit adjacent the housing. The at least a device may be a tube clip located on said external front-facing surface of the housing.

The bioreactor apparatus may further comprise a pumping arrangement configured to pump fluid through said fluid conduit. The pumping arrangement may comprise a peristaltic pump, preferably a linear peristaltic pump, located within a cavity of the bioreactor apparatus. The cavity may comprise a flexible membrane arranged between the peristaltic pump and the cell culture chamber, during use. The pumping arrangement may comprise a piezo driven valveless pump.

The bioreactor apparatus may further comprise a gas port on said external front-facing surface of the housing, the gas port arranged to be fluidly connected to a cell culture chamber disposed within said internal cavity, preferably wherein the gas comprises sterile air and/or carbon dioxide. Additionally, or alternatively, gas may be supplied to the internal cavity of the housing. In this way, gas may be provided to diffuse through a gas permeable membrane of the cell culture chamber.

The bioreactor apparatus may further comprise at least one moveable cover provided on said external front-facing surface of the housing, said cover arranged to cover the opening to said internal cavity when in a closed position. Said cover may be configured for automated movement between the closed position and an open position in which said internal cavity can be accessed via said opening. The cover may be arranged to thermally and/or optically seal the internal cavity when in its closed position. Each internal cavity may be further configured to receive a tray in a position below a cell culture chamber, such that the tray may collect leakage of fluid from the cell culture chamber. For example, the opening may have a first portion to receive the cell culture chamber and a second portion to receive the tray. The cover may be configured to move between one or more intermediate positions between the closed position and the open position. The bioreactor apparatus may further comprise one or more LEDs configured to disinfect the cavity. The cover may be configured to be moved to the closed position during operation of the one or more LEDs.

Said docking port may comprise an opening to an elongate slot or hole that extends into the housing.

The bioreactor apparatus may further comprise means for supplying heat to a cell culture chamber disposed in an internal cavity of the housing.

The housing comprise a plurality of internal cavities and/or a plurality of docking ports.

The housing may be configured to incubate individually each cell culture chamber contained within an internal cavity of the housing.

The bioreactor apparatus may further comprise means for agitating a cell culture chamber disposed within an internal cavity of the housing. The means for agitating the expansion chamber may comprise an ultrasound source, a vibration source, a means for rocking the expansion chamber, and/or a magnetic stirring means. In this way, it may be possible to re-suspend particles or cells in the expansion chamber. Furthermore, it may be possible to operate the bioreactor as a wave bioreactor, where the cell culture chamber is rocked during perfusion.

Also described herein is a bioreactor apparatus, comprising: a housing having at least one internal cavity for receiving therein a cell culture chamber; and an ultrasonic source for agitating a cell culture chamber within the housing.

Also described herein is a holder for a container (e.g. a consumable bag), comprising a substantially rectangular frame having a leg portion that extends down from a side of the frame, and then across substantially the width of the frame, in parallel to a lower portion of the frame.

A clip or tag for supporting the container within the frame may be provided on the frame, from which the container may be suspended. A tube clip may be attached to a side of the frame for retaining a portion of tube that is fluidly connected to a container, when mounted to the frame. The leg portion of the holder may be configured to be received within a correspondingly sized hole or bore in an apparatus, positioned such that a lower side of the frame may further rest on a surface of the apparatus.

It will be understood by a skilled person that any apparatus feature described herein may be provided as a method feature, and vice versa. It will also be understood that particular combinations of the various features described and defined in any aspects described herein can be implemented and/or supplied and/or used independently.

Moreover, it will be understood that embodiments are described herein purely by way of example, and modifications of detail can be made within the scope of the disclosure. Furthermore, as used herein, and “means plus function” features may be expressed alternatively in terms of their corresponding structure.

### BRIEF DESCRIPTION OF DRAWINGS

One or more embodiments will now be described, purely by way of example, with reference to the accompanying figures, in which:

FIGS. 1A and 1B show an embodiment of a cell culture chamber having a first housing and a second housing;

FIGS. 1C and 1D show a duct for transferring a sample from the second housing to a sampling loop in the first housing;

FIG. 1E shows a top view of a flow cell on a fluidic channel that may be used to inspect fluid travelling therethrough;

FIG. 1F shows an electrical sensor that may be embedded within a gas permeable membrane in the second housing of the cell culture chamber;

FIG. 1G shows a top view of the ports and fluidic channels in the first housing of the cell culture chamber;

FIGS. 2A and 2B show how the cell culture chamber of FIG. 1 may be operated for perfusion;

FIGS. 3A and 3B show how the cell culture chamber of FIGS. 1 and 2 may be operated during a sampling operation;

FIGS. 4A to 4D show different ways in which the gas permeable membrane may be secured to the bottom surface of the second housing;

FIGS. 5A and 5B depict an embodiment of a fluid manifold with fluid conduits for supplying fluid to the cell culture chamber shown in FIGS. 1 to 4;

FIG. 5C depicts the fluid manifold with fluid conduits for supplying fluid to a filtration device;

FIGS. 5D and 5E show an assembled view and an exploded view, respectively, of the fluid manifold;

FIG. 6A shows a tube matrix that may provide the fluid conduits of the manifold in FIGS. 5A to 5E;

FIG. 6B shows a sealing tool that may be used to seal the tube matrix in order to provide different fluid path configurations;

FIG. 6C shows an example of a fluid path configuration that may be formed by operating the sealing tool in the configuration shown in FIG. 6D;

FIGS. 7 and 8 show how a longer sealing tool may be used to seal multiple fluid conduits simultaneously;

FIGS. 9 and 10, show another embodiment of a tube matrix with an additional cross-conduit;

FIG. 11 shows two manifolds attached adjacent to each other;

FIG. 12A shows an embodiment of a bioreactor apparatus having at least one cavity for receiving a cell culture chamber;

FIG. 12B shows a holding device that may hold a container of fluid for connection with a cell culture chamber located within a cavity of the bioreactor apparatus;

FIGS. 12C to 12E show a cross-sectional side view, a cutaway plan view and a cutaway perspective view, respectively, of the bioreactor apparatus of FIG. 12A;

FIGS. 13A to 13D show a schematic diagram of internal components of the bioreactor apparatus;

FIG. 14A shows a tray that may detect moisture during a leak of the cell culture chamber; FIG. 14B shows a perspective view of the tray in combination with a cell culture chamber, and FIG. 14C shows a plan view of the cell culture chamber;

FIG. 14D shows how the tray may be inserted into the bioreactor apparatus of FIG. 12A;

FIGS. 15A to 15c show side views of a peristaltic pumping mechanism being connected to a cell culture chamber;

FIG. 16A shows a control interface connected to a bioreactor apparatus;

FIG. 16B shows a plurality of bioreactor apparatuses connected together, and controlled with a single control interface;

FIGS. 17 and 18 show an embodiment of a bioprocessing system, where a plurality of bioreactor apparatuses are held within a plurality of movable shelves;

FIG. 19 shows an alternative implementation of a bioprocessing system, where the shelves are located in an incubation room with a gantry robot; and

FIG. 20 shows a side view of the incubation room shown in FIG. 19, with the gantry robot moving between a first position and a second position.

### DETAILED DESCRIPTION

Typical bioreactors, consumables and bioprocessing systems may have a number of problems. Due to the need to maintain a closed system when handling all the cells, reagents and other fluids, conventional consumables include a complicated network of external tubing in order to interface with the bioreactor. This network of external tubing is very difficult to handle; particularly when using an automated handling means such as a robotic device, it may highly impractical and unreliable to engage the tubing in free space around the consumable and manipulate said tubing and consumable so as to correctly install it into the bioreactor and/or other equipment. The complexity of the consumables also means that they may be difficult to manufacture cheaply and may be unreliable.

Additionally, such consumables typically have a large footprint and thus are not space efficient when used within a bioprocessing system. In particular, since the bioreactors and consumables cannot be efficiently stored within a bioprocessing system, the bioprocessing system has a limited capacity for the number of cell therapy operations that may be performed at a time.

The complex tubing networks also mean that sampling from the consumables can be problematic, since the sample must often travel through a large length of tube (known as the “dead volume”) before reaching a sampling location. This dead volume is furthermore problematic, as when new reagents are added to the cell culture vessel, this volume must also be purged.

In addition, at least the following specific processes can be difficult to perform in bioreactors using existing consumables. Firstly, media is often stored externally at cool temperatures before being heated and injected into the culture chamber, since directly injecting into the culture chamber may slow cell growth. However, with existing systems, the heating may take a substantial amount of time due to the media needing to pass through long tubes inside ovens to heat up. Secondly, when gas may need to be added such as to oxygenate the media, this is typically achieved using a separate media conditioning chamber; this further complicates the consumable and requires further tubes and fluid connections to be provided. Thirdly, mixing of other reagents such as cytokines may be complicated, due to the need for further fluid connections to the consumable and the risk that addition of reagents may disturb or wash away molecules (cytokines) being generated by the cells themselves. Particularly, when all of the above processes need to be optimized to maximise T cell expansion, typical bioreactors struggle to effectively address the above problems simultaneously.

Therefore, it is desirable to provide an improved “consumable” cell culture chamber and bioreactor that address the problems discussed above, in particular with regards to distribution of fluid through tubing and reducing the form factor and dead volume required to perform all the necessary cell therapy steps.

In general, the present disclosure relates to expansion of cell cultures within a bioprocessing system 1. A typical cell therapy process may involve extracting cells from a patient, and subsequently expanding the cell culture to produce a larger number of cells for use in other steps in the process. The expansion of cells may take place in a cell culture chamber, such as the cell culture chamber 200 described herein. Various fluids (such as media, gas, cytokines, and/or cells) may be supplied to and removed from the cell culture chamber 200 during the expansion process. The expansion may take place in a bioreactor apparatus such as the bioreactor apparatus 100 described herein. The bioreactor apparatus 100 may incubate one or more cell culture chambers 200 contained therein and may provide means for supplying the various fluids to each cell culture chamber 200, and for analysis of the cells. The bioprocessing system 1 may contain a plurality of bioreactor apparatuses 100, which each may have a plurality of cell culture chambers 200. As will be described later in more detail, the bioprocessing system 1 may have a number of processing stations 2 for carrying out other steps in the cell therapy process. The bioprocessing system 1 may comprise an automated system with at least one robotic device 30 for manipulating the cell culture chambers 200, bioreactor apparatuses 100 and other components such as the processing stations 2. The robotic device 30 may have an automated mechanism such as a robotic end effector for manipulating the components of the bioprocessing system 1.

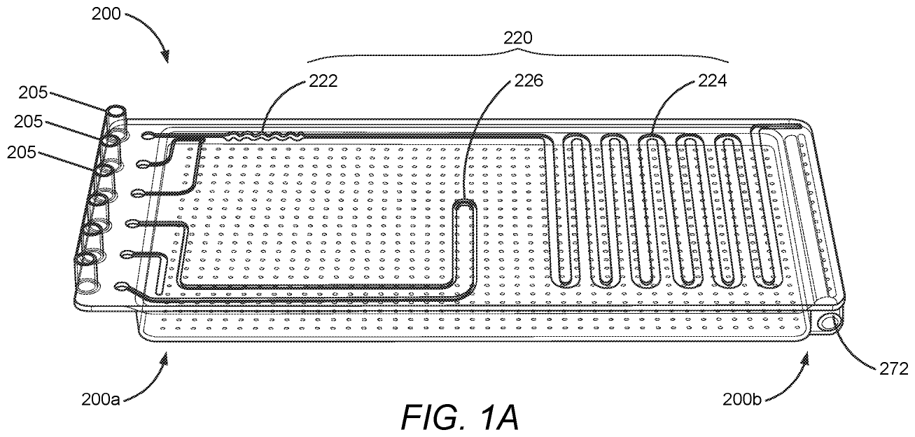

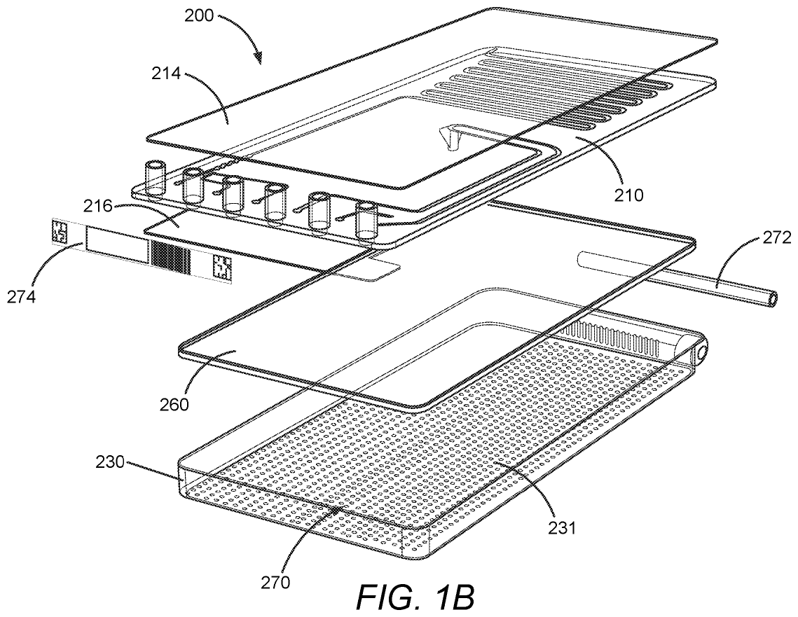

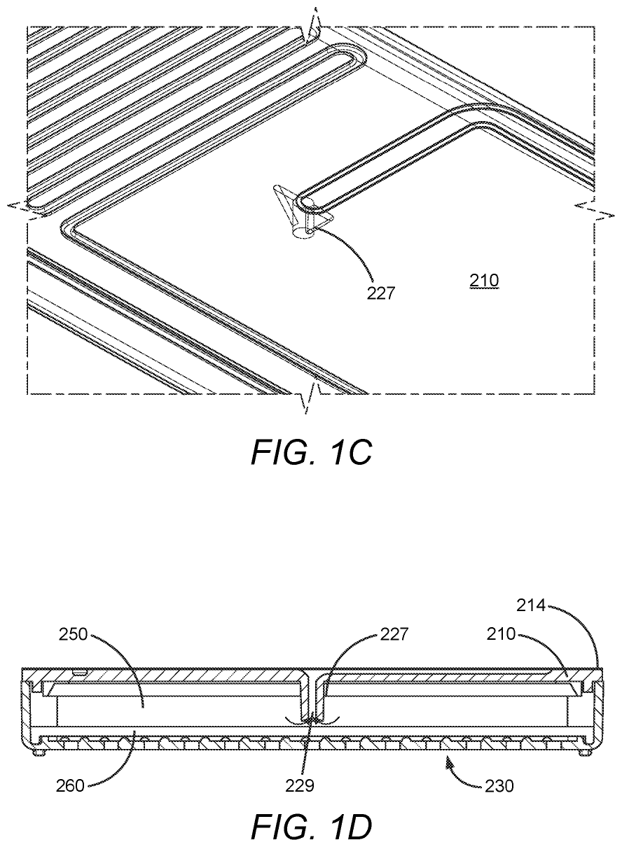

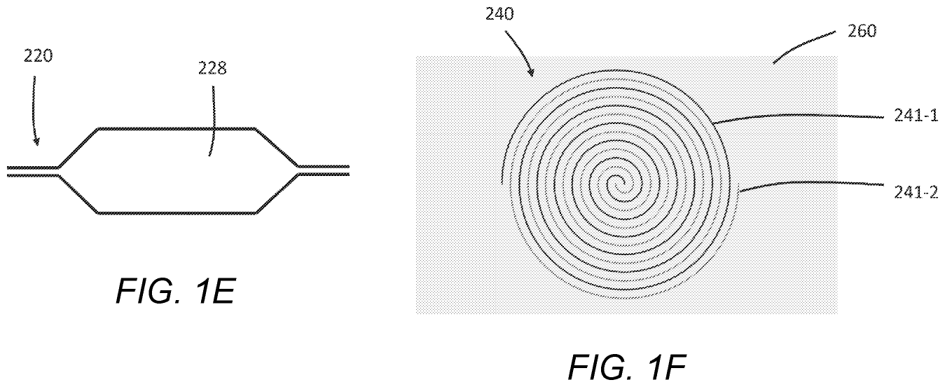

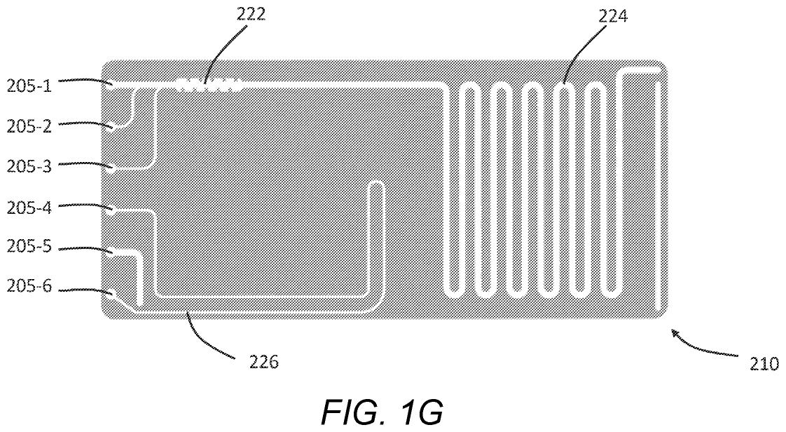

Referring to FIG. 1, and embodiment of the cell culture chamber 200 will now be described in detail. The cell culture chamber 200 may have a substantially cuboid shape, with a longitudinal direction extending between a first end 200a and a second end 200b. The cell culture chamber 200 may have a width (in a lateral direction) and a height (in a vertical direction). The cell culture chamber 200 comprises a plurality of ports 205, which may be used for input and output of fluid. As used herein the term fluid may refer to both liquid and gas; for example, fluid may refer to a liquid medium 250, cells 251, gas or air 252, cytokines 253, waste, and/or samples 255, and mixtures thereof. The ports 205 are preferably arranged in a linear configuration, and preferably on a common surface of the cell culture chamber 200. In this example, the ports 205 are arranged at the first end 200a of the cell culture chamber 200. In this way, fluid connections to the cell culture chamber 200 may be standardised and simplified.

The cell culture chamber 200 comprises a first (e.g. upper) housing 210. The first housing 210 has an upper surface and a lower surface, with one or more open fluidic channels 220 formed on at least one of the upper and lower surfaces. At least one sealing layer 214, 216 is arranged to be affixed (e.g. laminated) onto at least one of the upper or lower surfaces of the first housing 210, the sealing layer 214, 216 arranged to seal said one or more open fluidic channels 220. In this example, the open fluidic channels 220 (e.g. slots) are moulded into the first housing 210, with a first sealing layer 214 being affixed (e.g. laminated) onto the upper surface of the first housing 210 and a second sealing layer 216 being affixed (e.g. laminated) onto the lower surface of the first housing 210. In this way, closed fluidic paths 220 are provided through the first housing 210. Alternatively, grooves may be formed onto only one (upper or lower) surface of the first housing 210 which may then be sealed by a single sealing layer 214, 216. However, by providing two sealing layers 214, 216, the moulding of the first housing 210 may be simplified, and it may be easier to have more functionality embedded into the cell culture chamber 200, for example having a flow cell to image the cells on the upper surface, and the heat exchange portion on the lower surface. Examples of a flow cell 228 and a heat exchange portion 224 will be described in detail further on. The closed fluidic paths 220 may be provided by the first housing 210 in combination with both sealing layers 214, 216; for example, a through-hole filter may be provided in the first housing 210 where an input path on one surface of the first housing 210 leads to an output path on the other surface of the first housing 210 via the through-hole filter. Alternatively or additionally, corresponding (i.e. matching or overlapping) fluidic paths 220 may be provided on both the upper and lower surfaces of the first housing 210, thereby increasing the rate of fluid flow through the first housing 210. Preferably, the minimum width of the fluidic channels 220 is less than 3 mm and more preferably less than 1 mm; in this way, the dead volumes of the fluidic channels 220 may be reduced. In this configuration, the fluidic channels 220 may be referred to as “millifluidic channels” 220.

As an alternative construction of the first housing 210, it may be possible to create the same structure with a first housing 210 containing fluidic paths 220 by manufacturing methods including diffusion bonding. In the example of diffusion bonding, channels would be milled directly into a plastic surface which would then be diffusion bonded onto another surface; multiple layers could be stacked and bonded together in order to achieve a complex fluidic routing within a relatively simple part. This is advantageous as compared to the laminated route discussed above, as multiple layers of fluidic paths 220 can be bonded together. As a further alternative, the first housing 210 could be created by 3D printing, where the whole first housing 210 could be created as a single part with fluidic paths 220 directly printed into the first housing 220. Advantageously non-planar fluidic channel profiles could be incorporated using this method.

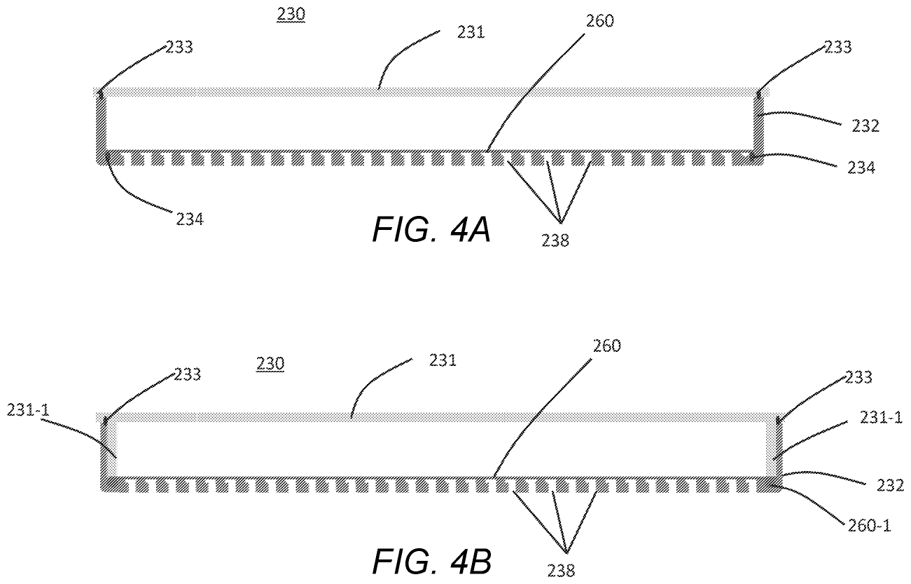

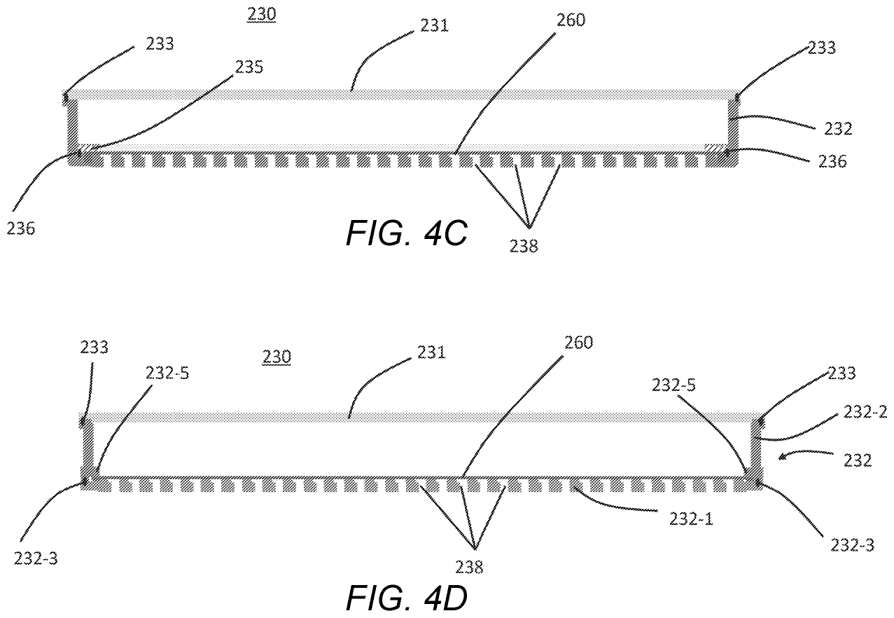

The cell culture chamber 200 also comprises a second (e.g. lower) housing 230. The second housing 230 is configured to house the cells 251 during incubation and expansion. A bottom surface of the second housing 230 may have a porous structure such as a plurality of perforations or openings 238. As will be described further with respect to FIGS. 4A to 4D, a gas permeable membrane 260 may be fixed onto the bottom surface of the second housing 230 to facilitate transfer of gas into the second housing 230 while preventing leakage of fluid from the second housing 230. The gas permeable membrane 260 may be about 100 μm thick. The gas permeable membrane 260 may comprise silicone. A gas permeable membrane 260 is particularly advantageous for culturing large numbers of T cells. Alternatively, the cell culture chamber 200 may not include a gas permeable membrane 260. For example, the bottom surface of the second housing 230 may comprise a (non-gas-permeable) tissue culture polystyrene, which may be particularly advantageous for culturing adherent cells. Where a gas permeable membrane 260 is not provided, gas may diffuse to cells 251 from above the media 250 that is located in the second housing 230.

The first housing 210 and the second housing 230 may be joined together, such as by using an ultrasonic welding technique. Alternatively, the first housing 210 may be joined to the second housing 230 using adhesive, solvent bonding and/or diffusion bonding. Alternatively, the first housing 210 and the second housing 230 may be clamped together via an O-ring seal, such as in the manner described in relation to FIG. 4B. The join between the first housing 210 and the second housing 230 preferably forms a fluidic seal therebetween. The cell culture chamber 200 may have an engagement feature arranged to be engaged by an automated mechanism whereby to facilitate manipulation of the cell culture chamber 200 by the automated mechanism. The automated mechanism may be the robotic device 30 in the bioprocessing system 1. The engagement feature may be provided by a portion of the first housing 210 extending beyond the second housing 230, e.g. to form a protruding portion or element that can be engaged or gripped by an automated mechanism.

The cell culture chamber 200 may comprise a pH indicator 270 arranged such that the pH of a fluid contained in the second housing 230 can be determined via inspection of the pH indicator 270. The inspection of the pH indicator 270 may be a visual inspection, such as by monitoring a change in colour, but the inspection may refer to monitoring other properties. For example, the pH indicator 270 may indicate pH of the fluid based on fluorescence lifetime. In this example, the pH indicator 270 may be a pH label 270 located on the second housing 230, though the pH label 270 may be placed elsewhere in the cell culture chamber 200. Furthermore, additional pH indicators 270 may be located at different locations in the cell culture chamber 200.

The cell culture chamber 200 may comprise an identification mark 274. The identification mark 274 may be a label to uniquely identify the cell culture chamber 200. The identification mark 274 may display patient information, and may include a QR code, barcode, or any other suitable mark for identifying the cell culture chamber 200. The identification marks 274 may be monitored by a machine vision system in the bioprocessing system 1 in order to track the location of each cell culture chamber 200. The identification mark 274 may be located anywhere on the cell culture chamber 200 suitable for inspection by a human operator and/or a machine vision system.

The cell culture chamber 200 may comprise a means for pumping fluid through the closed fluidic paths 220. The means for pumping may comprise a flexible tube 272 connected to the closed fluidic paths 220. The flexible tube 272 is arranged to interface with a peristaltic pump located in the bioreactor apparatus 100; for example, the flexible tube 272 may be located at the second end 200b of the cell culture chamber 200. The peristaltic pump may be configured to form a compressed portion of the flexible tube 272, and subsequently move the compressed portion along the length of the flexible tube 272 in a pumping direction, thereby forcing fluid through the flexible tube 272. The peristaltic pump is preferably a linear peristaltic pump, but other configurations (such as rotary peristaltic pumps) may be used. Peristaltic pumps may be particularly advantageous since they allow control of the volume of fluid being pumped. Alternatively, the cell culture chamber 200 may comprise an integrated peristaltic chip arranged to interface with an external driver. For example, in addition to the flexible tube 272, the cell culture chamber 200 may comprise other components of a peristaltic pump arranged around the flexible tube 272, where an external driver drives motion of the peristaltic pumping components. In other words, the cell culture chamber 200 at least partially comprises a peristaltic pump which may be externally driven (such as by the bioreactor apparatus 100).

The cell culture chamber 200 may comprise a mixer 222 for reagent or gas exchange. In this embodiment, as shown particularly in FIGS. 1A and 1G, the fluidic paths 220 may comprise an in-channel mixing portion 222. For example, this may be a fluidic path 220 comprising obstructions that cause the flow to split, fold and recombine into each other and achieve laminar mixing. The mixer 222 may be implemented in other ways, which may use either passive or active micromixing techniques. Examples of passive micromixing techniques include: lamination structures, zigzag channels, 3D serpentine structures, embedded barriers, twisted channels, and/or surface-chemistry techniques. Examples of active micromixing techniques include: acoustic/ultrasonic, dielectrophoretic, electrokinetic time-pulsed, electrohydrodynamic force, thermal actuation, magneto-hydrodynamic flow, electrokinetic instability. Any mixing techniques may be provided in any suitable combination to provide the mixer 222.

The fluidic paths 220 may also comprise a heat exchange portion 224. For example, the heat exchange portion 224 may comprise a winding pathway within a region of the first housing 210. The first housing 210 may include a heating element (not shown) for heating fluid in the heat exchange portion 224. For example, the heating element may be a PCB or heating coil mounted to the top of the first housing 210. Alternatively, a heating element may be located within a bioreactor apparatus 100 at a position that is adjacent to the heat exchange portion 224 when the cell culture chamber 200 is inserted into a bioreactor apparatus 100. It is advantageous for media to be heated prior to entry into second housing 230 since addition of media at a lower temperature may affect growth of the cells 251 within the second housing 230. By providing a heat exchange portion 224 in the first housing 210, media can be stored externally to the cell culture chamber 200 at room temperature, and only heated when transferred into the cell culture chamber 200. This may reduce the energy required to maintain external containers at an increased temperature, and may allow the containers to be refrigerated, thereby increasing their lifetime.

The fluidic paths 220 may also comprise a sampling loop 226, from which a sample can be extracted from the cell culture chamber 200. The sampling loop 226 may be connected to two fluid ports 205 of the cell culture chamber 200. As shown in FIGS. 1C and 1D, and the sampling loop 226 may be connected to a duct 227 that provides a sampling channel 229 into the fluid 250 contained in the second housing 230. By providing a sampling loop 226 within the fluidic paths 220 of the cell culture chamber 200, the dead volume is substantially reduced compared to existing sampling methods that pump a sample into external tubing. Having a low dead volume may reduce the waste of fluid 250 during a sampling operation.

The fluidic paths 220 preferably include a means for inspecting the fluid, such as a flow cell 228. The flow cell 228 may be a region in the fluidic paths 220 with a reduced thickness or depth, thereby spreading out the cells 251 in the first housing 210. FIG. 1E depicts a top view of a flow cell 228, where the width of the fluidic path 220 has widened in conjunction with a decrease in the depth of the fluidic path 220. In this way the cells 251 may be more easily counted or otherwise analysed, such as by a microscope, or by dynamic light scattering, capacitance, impedance, and/or spectroscopy measurements such as by using Raman spectroscopy (for metabolite sensing measurements). The flow cell 228 may be implemented in a recirculation loop (not shown) of the cell culture chamber 200. Preferably, the flow cell 228 is located on the sampling loop 226, though the means for inspecting the fluid may be located anywhere in the cell culture chamber 200. For example, a transparent window located on the second housing 230 may enable an approximate cell count to be determined based on optical transmission of the layer of cells 251, or of resuspended cells 251 circulating through a transmission measurement chamber, i.e. a separate fluidic compartment formed by the joining of the first housing and the second housing (not shown). The transmission measurement chamber may operate in a similar manner to the flow cell 228 in the first housing 210, but since the transmission measurement chamber is located in the second housing 220 it may be possible to view a wider depth of cells 251, which may be useful for looking at a large cell population. The measurements taken by the means for inspecting may be used to adjust operation parameters of the cell culture chamber 200 and/or the bioreactor 100. These operation parameters may include the perfusion rate of fluid through the second housing 230, and/or the rate of gas supply to the gas permeable membrane 260.

The cell culture chamber 200 may comprise means for filtering washing and/or measuring cell count (not shown). For example, the fluidic paths 220 may include a portion arranged for microfluidic or millifluidic cell separation and sorting. This may comprise one or more of: filters, hydrodynamic structures, deterministic lateral displacement structures, field-flow fractionation structures, microstructures such as grooves, chevrons, and/or herringbones, an inertial separation and sorting portion, a gravity and sedimentation separation and sorting portion, a biomimetic separation and sorting portion, a magnetophoresis separation and sorting portion, an aqueous two-phase system, an acoustophoresis cell separation and sorting portion, and/or a dielectrophoresis cell separation and sorting portion. Non-microfluidic techniques may be used for filtering, such as by including at least one hollow fibre filter, which may be in-line with an outlet of the cell culture chamber 200.

Since the microfluidic techniques and millifluidic channels 220 discussed herein may typically be suitable for systems with much smaller overall volumes, such techniques may not appear suitable for incorporation into cell therapy systems which typically require volumes of fluid from 10 mL up to a few Litres. Indeed, most existing cell culture consumables use tubing so that the fluid may be controlled using pinch valves and peristaltic pumps.

The cell culture chamber 200 may comprise an electrical sensor 240 configured to measure impedance and/or capacitance of a fluid within the cell culture chamber. As depicted in FIG. 1F, the electrical sensor 240 may be embedded within the gas permeable membrane 260, thereby locating it in close proximity to the fluid in the second housing 230. The electrical sensor 240 may comprise a plurality of electrodes 241, which may be formed from metal. Where the electrodes 241 are metal, the electrodes 241 may have a thin profile so as not to interfere with gas diffusion through the gas permeable membrane 260. Alternatively, the electrodes 241 may be formed from patterned conductive silicone, which may be filled with carbon nanotubes. The electrodes 241 may also provide structural rigidity to the gas permeable membrane 260. In FIG. 1F, the electrical sensor 240 comprises a first electrode 241-1 and a second electrode 241-2 in a double spiral arrangement. In a similar manner to with a rolled capacitor, arranging the electrodes 241 in a double spiral arrangement allows the electrical sensor 240 to have a reduced form factor while still providing a large length of each electrode 241 on the gas permeable membrane 260.

FIG. 1G depicts a plan view of the first housing 210 showing the ports 205 and fluidic paths 220. In this example, six ports 205 are present, though in other examples there may be more or fewer ports 205. A first port 205-1 is configured as an input for the medium 250. A second port 205-2 is configured as an input for cytokines 253. A third port 205-3 and a fourth port 205-4 are configured as inputs for air 252, such as a mixture of CO₂ and air. The fluidic channels 220 connecting to the first port 205-1, second port 205-2 and third port 205-3 merge together prior to the mixer 222.

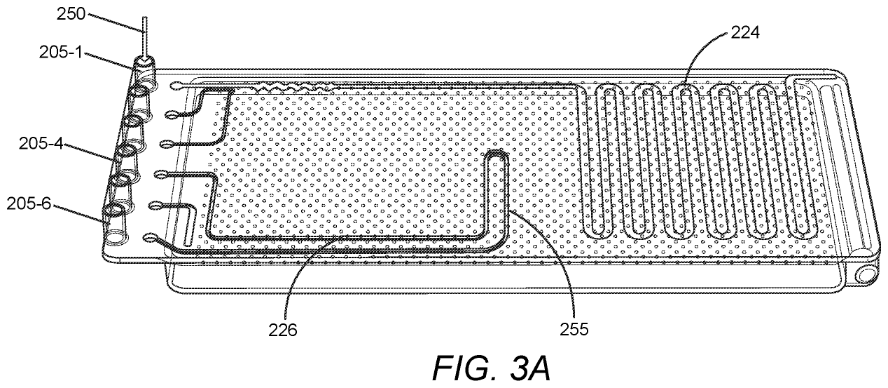

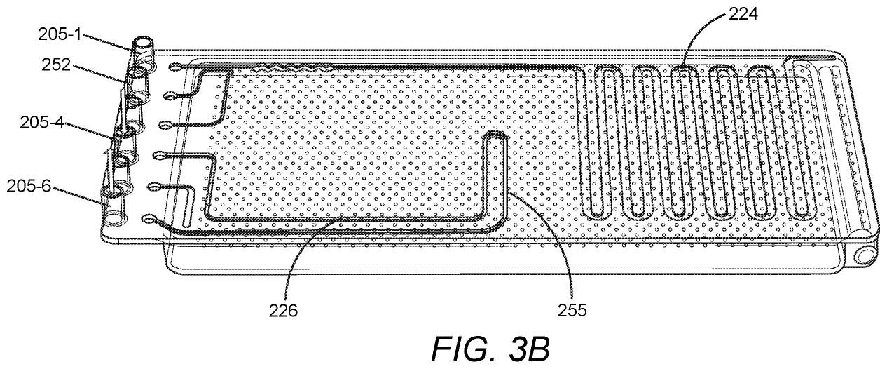

A fifth port 205-5 is configured as an output for waste, such as medium 250 that has flowed from the first port 205-1 into the second housing 230, and then back through the second housing 230 during perfusion. A sixth port 205-6 is configured as an input or output port for cell samples 255. The sampling loop 226 is connected to the fourth port 205-4 and the sixth port 205-6. As will be described in relation to FIGS. 3A and 3B, this allows a sample 255 to be extracted from the second housing 230 via addition of air 252 to the fourth port 205-4. While this embodiment provides the ports 205 in this particular configuration with the above-described functions, it will be appreciated that the ports 205 may be provided in any order and may provide different functions. Furthermore, multiple ports 205 may be provided for a particular function; for example there may be multiple input/output ports, which may be connected to a respective mixer 222.

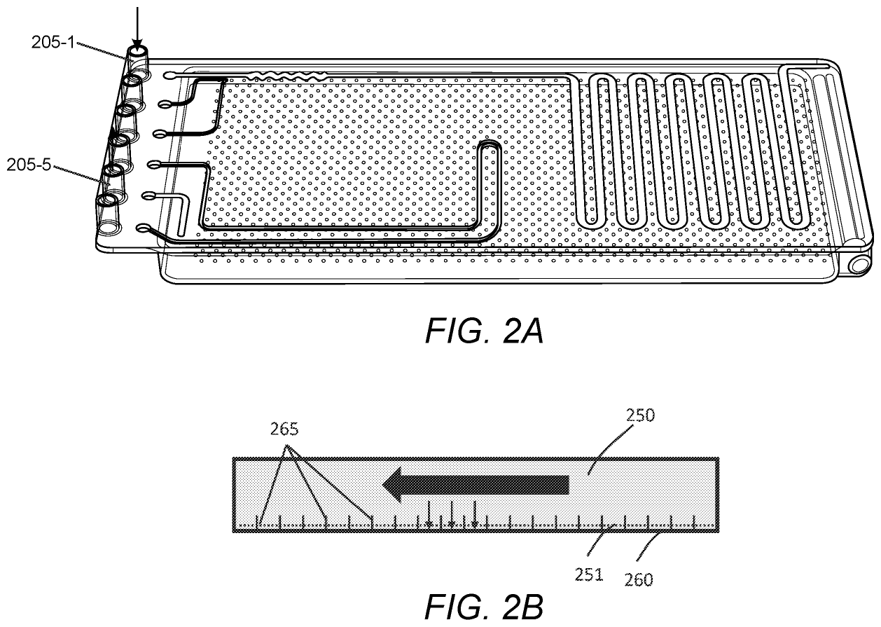

The cell culture chamber 200 may be operated in perfusion, as shown in FIGS. 2A and 2B. For perfusion, a medium 250 is supplied continuously through the cell culture chamber 200 such as into the first port 205-1, as indicated by the arrow. The medium 250 is supplied from an external container, which may be stored at ambient temperature. The medium 250 flows through the mixer 222 and the heat exchange portion 224 where the medium 250 is warmed by a means for heating, such as a heater located in the bioreactor assembly 100. The medium 250 may subsequently pass into the second housing 230 via a splitter at the second end 200b of the cell culture chamber 200. The splitter distributes the flow of medium 250 so that it is supplied substantially uniformly across the width of the second housing 230. For example, the splitter may be a wall or pipe with a plurality of openings distributed across the width of the second housing 230. The medium 250 subsequently flows through the second housing 230 in a longitudinal direction from the second end 200b to the first end 200a. The flow of medium 250 facilitates growth of cells 251 at the bottom of the second housing 230, such as a layer of cells 251. A plurality of baffles 265 may be located on the bottom of the second housing 230, such as on the gas permeable membrane 260 in order to prevent the cells 251 from being washed away by the flow of medium 250. Other methods to reduce disturbance of cells 251 may also be used, such as by adjusting flowrates, and modifying the geometry of the second housing 230.

At the first end 200a of the cell culture chamber 200, the medium 250 flows out of the fifth port 205-5, where it may be collected in a waste container. The flow of medium 250 may be facilitated by a means for pumping, such as by operation of a peristaltic pump in the bioreactor apparatus 100 on the flexible tube 272. Alternatively, there may be a pressure difference provided between the first port 205-1 and the fifth port 205-5, thereby forcing the medium 250 through the cell culture chamber 200.

The cell culture chamber 200 may have a substantially planar configuration, where the ratio between the length of the second housing 230 to the height of the second housing is at least 1:1 and more preferably at least 4:1. In this way, the flow of medium 250 remains shallow, so that the medium 250 remains in close proximity to the cells 251 at the bottom of the second housing 230. Furthermore, cells 251 may more rapidly resettle following an agitation or resuspension process. By having a large length of the second housing 230, the medium 250 may travel across a large area of the second housing 230, thereby supplying medium 250 to a large number of cells 251. In other words, having a large ratio of length to height of the second housing 230 allows good perfusion of nutrients across the layer of cells 251, and the amount of medium 250 that enters and exits the second housing 230 near its top surface without reaching the cells 251 is reduced.

A sampling operation will now be described in relation to FIGS. 3A and 3B. First, both the fourth port 205-4 (for air) and the fifth port 205-5 (for waste output from the second housing 230) are closed. The sixth port 205-6 is left open. Preferably, the second housing 230 may be agitated in order to mix the cells 251 from the cell layer into the medium 250; ways in which the second housing 230 may be agitated will be described later in further detail. Preferably, an air layer is maintained at the top of the second housing 230 to allow free movement of the medium 250 in the second housing 230. Subsequently, as shown in FIG. 3A, medium 250 is added to the first port 205-1, where it gets heated in the heat exchange portion 224 before entering the second housing 230. The medium 250 may be pumped into the second housing 230 using the peristaltic pump. Since the fifth port 205-5 is closed, this increases pressure in the second housing 230, which causes the medium 250 (and any cells 251 suspended therein) to be forced up the sampling channel 229 of the duct 227 and into the sampling loop 226. Flow of medium 250 into the first port 205-1 may be maintained as long as necessary to provide the required volume of a fluid sample 255 in the sampling loop 226. Once this has occurred, the flow of medium 250 in the first port 205-1 is stopped. Subsequently, as shown in FIG. 3B, air 252 is then added into the sampling loop 226 via the fourth port 205-4, which forces the sample 255 through the sampling loop 226 towards the sixth port 205-6 where the sample 255 may be collected for analysis. The sample 255 may be collected in a sampling container, which, as will be described later in more detail, may be mounted to a bioreactor apparatus 100 containing the cell culture chamber 200.

Extracting a sample 255 in this way is particularly advantageous, since the sample 255 does not need to travel through a substantial length of tubing in order to be extracted from the cell culture chamber 200. This limits the amount of fluid that is wasted during extraction from the cell culture chamber 200 and reduces the excess gas that may accumulate within a sampling container during a sampling operation. Furthermore, the volume of the sample 255 can be controlled simply by addition of the medium 250 to the second housing 230.

As discussed previously, the second housing 230 may have a plurality of openings 238 on its bottom surface, with a gas permeable membrane 260 on the bottom surface to allow gas to reach the cells 251. In order to prevent leakage of fluid out of the second housing 230, the gas permeable membrane 260 needs to be fixed to the bottom surface of the second housing 230. Several ways in which this may be achieved will now be described in relation to FIGS. 4A to 4D.

In FIG. 4A, the second housing 230 comprises a lid 231, and a tray 232, which are connected together such as with one or more heat welds 233. Preferably, the lid 231 is provided by the first housing 210 of the cell culture chamber 200. Alternatively, an ultrasonic join 233 may be used. The gas permeable membrane 260 is attached to the bottom of the tray 232 using glue 234, which may be applied around the perimeter of the base of the tray 232. Advantageously, this requires a small number of components. However, there is an additional gluing step, and it may be difficult to perform quality control of the liquid seal between the gas permeable membrane 260 and the bottom of the second housing 230.

In FIG. 4B, the lid 231 comprises at least one protruding portion 231-1 that extends towards the base of the tray 232, thereby clamping the gas permeable membrane 260 to the bottom of the tray 232. Preferably, the lid 231 is provided by the first housing 210 of the cell culture chamber 200. An O-ring 260-1 may be located at the base of the tray 232 and may be provided by the gas permeable membrane 260. As with the embodiment in FIG. 4A, the lid 231 may be connected to the tray 232 using one or more heat welds 233 or an ultrasonic join 233. Alternatively, the lid 231 may be connected to the tray 232 by a snap feature, with the fluidic seal provided by the O-ring 260-1. Advantageously, this only requires to parts that may be easily injection moulded, and now additional steps (such as gluing) are required to form the seal. However, it may be challenging to maintain a consistent seal without blocking inlets and outlets to the second housing 230. Furthermore, the increased distance between the heat welds 233 and the clamping of the gas permeable membrane 260 to the bottom of the tray 232 may create difficulties with clamping force tolerance control.

In FIG. 4C, the lid 231 is connected to the tray 232 with one or more heat welds 233. Preferably, the lid 231 is provided by the first housing 210 of the cell culture chamber 200. The second housing 230 further comprises inner frame 235, which extends around the perimeter of the interior of the tray 232. The inner frame 235 is connected to the tray 232 with one or more heat welds 236, thereby clamping the gas permeable membrane 260 to the bottom of the tray 232. Advantageously, this attaches the gas permeable membrane 260 to the bottom of the tray 232 without obstructing any fluid inlets and outlets to the second housing 230. However, this requires an additional component (the inner frame 235) and an additional welding step. Furthermore, this may slightly decrease the area within the second housing 230 available for growing cells 251.

In FIG. 4D, the tray 232 comprises a base portion 232-1 and a side portion 232-2. The lid 231 is attached to the side portion 232-2 by one or more heat welds 233. Preferably, the lid 231 is provided by the first housing 210 of the cell culture chamber 200. The side portion 232-2 is attached to the base portion 232-1 by one or more heat welds 232-3. The side portion 232-2 comprises at least one internal protrusion 232-5 that extends over the gas permeable membrane 260, thereby clamping the gas permeable membrane 260 to the base portion 232-1 of the tray 232 when the side portion 232-2 is attached to the base portion 232-1. Advantageously, it is possible to achieve clamping of the gas permeable membrane 260 to the base portion 232-1 without decreasing the area available for growing cells 251. Furthermore, the clamping point is close to the welding point, which may help with clamping force tolerance control. However, this requires an additional component and an additional welding step.

The cell culture chamber 200 described herein has a number of advantages. Since all of the fluidic routing and pumping elements are moulded in a simple ultrasonically welded construction, the cell culture chamber 200 has a small footprint, has a large number of features and is easy to install within a bioreactor apparatus 100. In other words, by integrating the fluidic paths 220 onto a single planar housing 210, the bill of materials, assembly costs, and/or the overall size of the cell culture chamber 200 may be reduced. Additionally, it is straightforward to create variants of the cell culture chamber 200 with different fluidic paths 220 simply by replacing the first housing 210.

Furthermore, the cell culture chamber 200 allows integrated mixing, heat exchange and sampling, which may previously have required separate containers and/or tubes. By providing a mixer 222 within the cell culture chamber 200, the medium 250, cells 251, air 252, and/or cytokines 253 may be easily mixed together, without needing to make further fluid connections to separate containers or provide additional mixing devices. The built-in heat exchange portion 224 means that the medium 250 does not need to be maintained at an increased temperature before addition into the cell culture chamber 200. This reduces overall energy usage and increases the lifetime and shelf-life of the medium 250. By including a sampling loop 226, a sample 255 may be readily extracted from the cell culture chamber 200 simply by operating the means for pumping and/or addition of air 252 into one of the ports 205; operating the sampling loop 226 in this way also allows the volume of the sample 255 to be controlled. As previously discussed, this sampling configuration has a very low dead volume, thereby decreasing waste of medium 250 and/or cells 251. By providing millifluidic channels through the first housing 210, the cell culture chamber 200 promotes efficient heat exchange, gas exchange and mixing due to the high surface area to volume ratios.

By having a large ratio between the length of the second housing 230 to the height of the first housing 230, there is a higher yield per cm², a reduced cost of the cell culture chamber 200, and a reduced energy required to incubate the cells 251 in the cell culture chamber 200. The cell culture chamber 200 also has separate supplies of medium 250 and gas to the cells 251 on the gas permeable membrane 260. This means that the supplies may be independently controlled to perfuse the medium 250 at whatever rate required for nutrient and waste exchange without affecting gas consumption. In addition, the splitter provides perfusion inlets that evenly spread the flow and diffuse into the second housing 230 from the top, such that a boundary layer will form in the bottom of the second housing 230 where the cells 251 are located, thereby inhibiting the cells 251 and/or cytokines 253 from being washed away. Since the means for agitating may also include a rocker, the cell culture chamber 200 may be operated as a wave bioreactor, thereby allowing further mixing for achieving high cell densities. In existing bioreactors, providing both sufficient mixing and gas and nutrient transport to the cells has been challenging without disturbing cells and/or washing away cytokines. The cell culture chamber 200 is includes a means for inspecting the fluid, thereby facilitating more precise nutrient control.

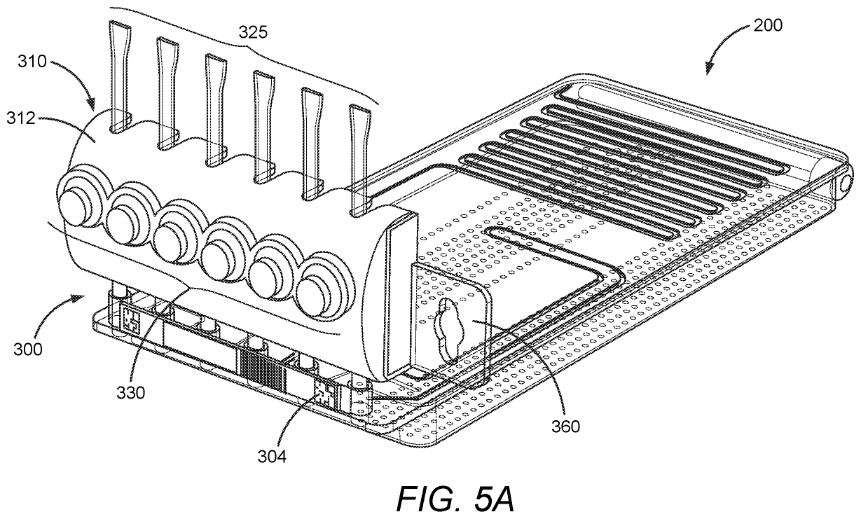

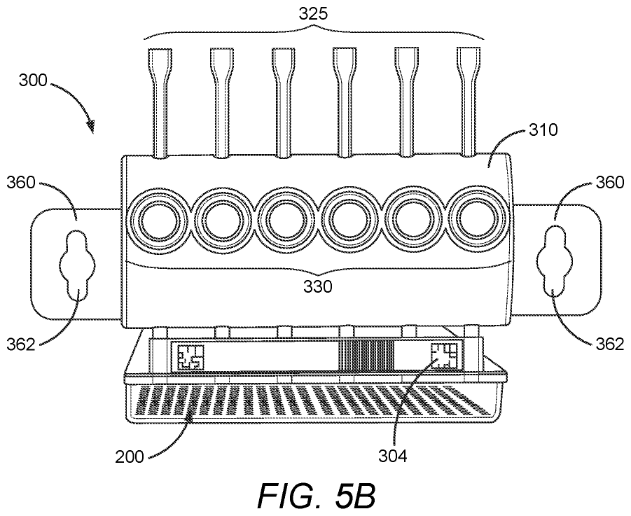

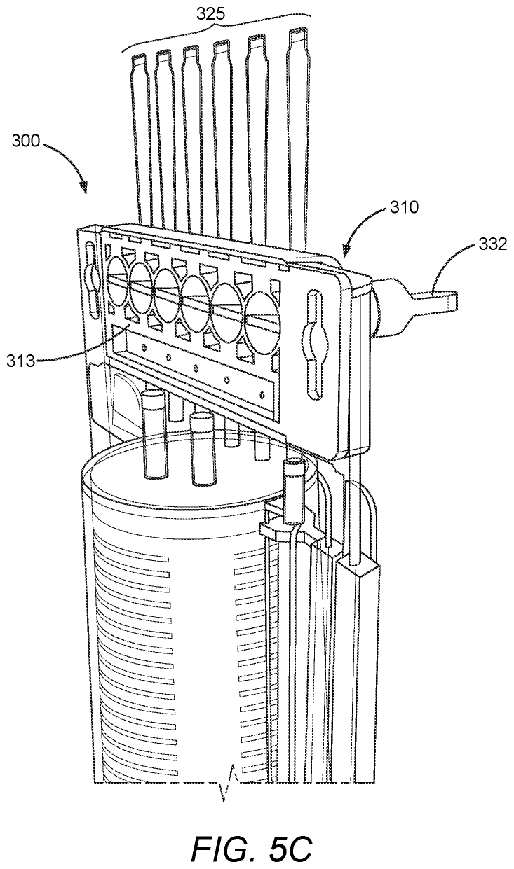

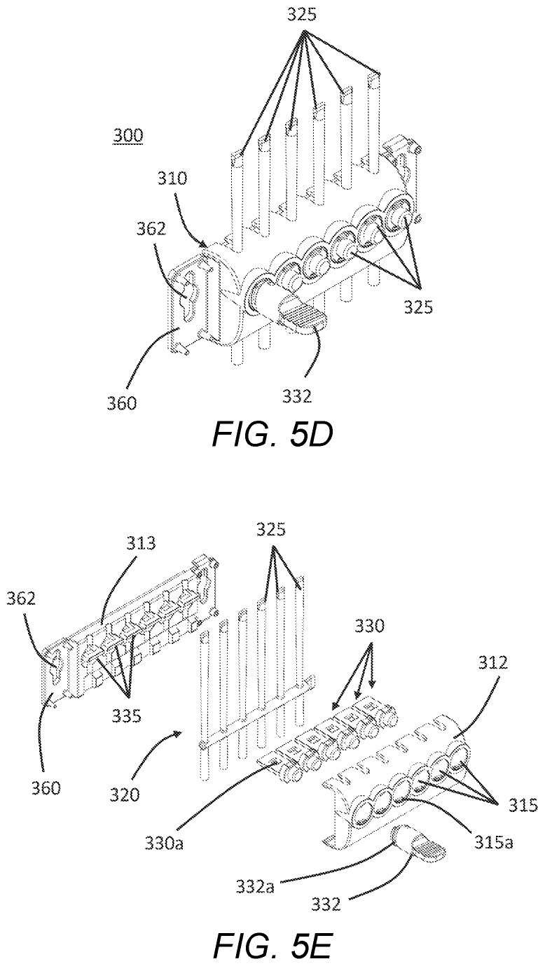

Referring now to FIG. 5, a manifold 300 will now be described. The manifold 300 is arranged to supply fluid to a bioprocessing apparatus. As shown in FIGS. 5A and 5B, the manifold 300 may supply fluid to the cell culture chamber 200 described previously. Alternatively, the manifold 300 may provide fluid to other apparatus, such as a filtration device as shown in FIG. 5C.

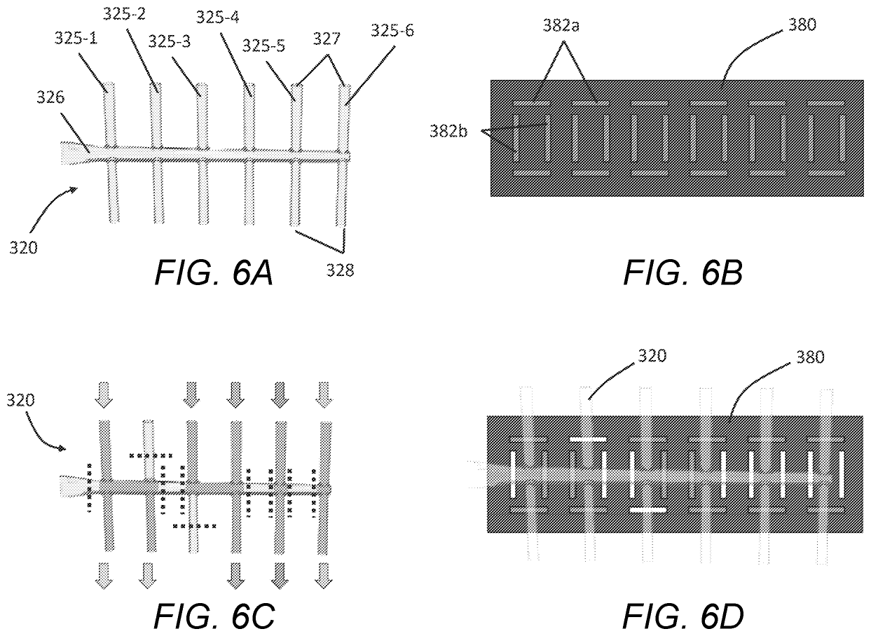

The manifold 300 comprises a housing 310. The housing 310 has a front portion (side) 312 arranged to be manipulated by a human operator, and a rear portion (side) 313 arranged to be operated by an automated means, such as a valving actuator 172 located on the bioreactor apparatus 100. The housing 310 may comprise an identification mark 304 to allow the manifold 300 to be uniquely identified by a machine vision system. Extending through the housing 310 are a plurality of flexible fluid conduits 325, with each fluid conduit 325 configured to be fluidly coupled to a corresponding port 205 provided on the bioprocessing apparatus. As shown in FIG. 5, the manifold 300 comprises six fluid conduits 325, each connected to the ports 205 on the cell culture chamber 200. The connection between the fluid conduits 325 and the ports 205 may be formed by tube welding. As shown in FIG. 5E, the front portion 312 may be removable from the rear portion 313, so that the flexible fluid conduits 325 may be installed into the housing 310. Preferably, the fluid conduits 325 are provided by a tube matrix 320 as described further in relation to FIGS. 6 to 10.

The manifold 300 also comprises valves 330 each configured to facilitate control of the flow of fluid through each of the fluid conduits 325. The valves 330 may be bidirectional pinch valves 330, where flow of fluid through each fluid conduit 325 is inhibited by pinching the fluid conduit shut 325. Each pinch valve 330 may have an opening 330a that the corresponding fluid conduit 325 passes through. In this way, movement of the pinch valve 330 relative to the fluid conduit 325 pinches the fluid conduit 325 shut. Preferably, the valves 330 may be operated by both a human operator and an automated means.

The front portion 312 of the housing 310 may comprise a plurality of valve apertures 315 corresponding to each of the valves 330. A human operator may control each of the valves 330 with a key 332 from the front portion 312. The key 332 has external threading 332a that engages with internal threading 315a on each of the valve apertures 315. In this way, a user may screw the key 332 into the valve aperture 315 thereby pressing the pinch valve 330 into the corresponding fluid conduit 325 and pinching said fluid conduit 325 shut.

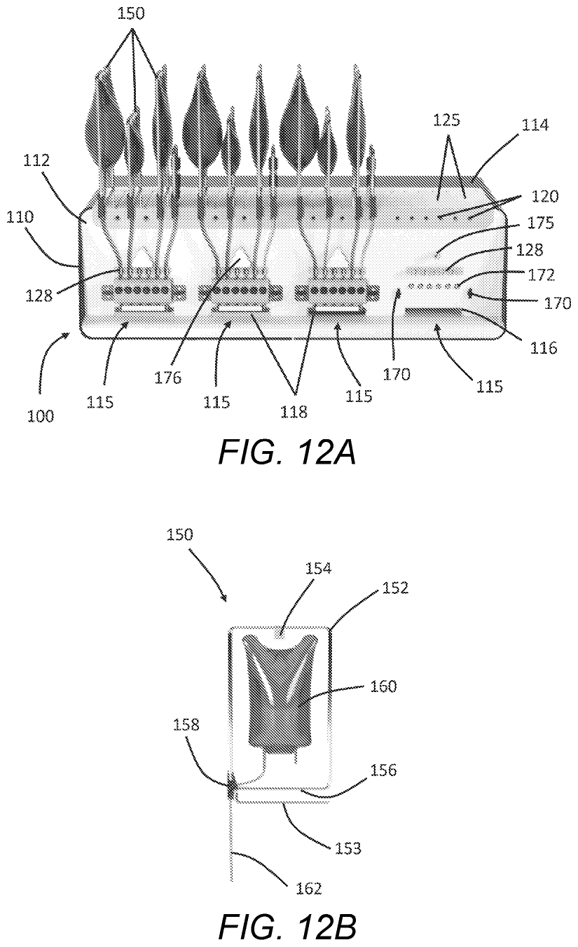

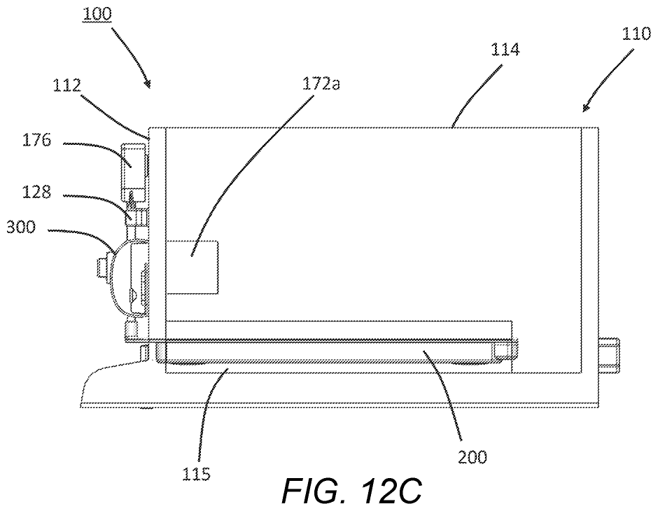

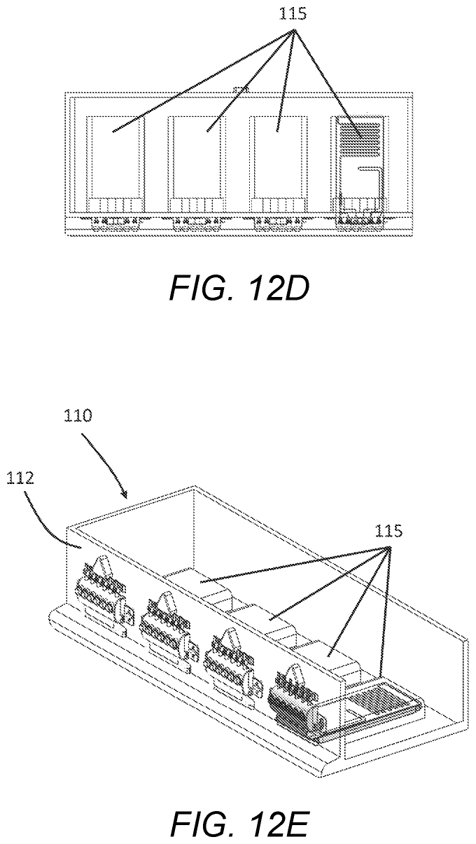

The rear portion 313 of the housing 310 may have openings 335 corresponding to each of the valves 330. When the manifold 300 is inserted into an apparatus such as the bioreactor apparatus 100, a valving actuator 172 on the bioreactor apparatus 100 may be operated to extend into each of the openings 335 thereby pinching shut a fluid conduit 325 that passes through each valve 330. The valving actuators 172 will be described in more detail in relation to FIG. 12. In this way, each valve 330 may also be operated by an automated means.

By providing the valves 330 on a manifold 300 (rather than being built into the bioreactor apparatus 100), a number of advantages are provided. Firstly, the valves 330 may easily accessible external to the bioreactor apparatus 100, thereby allowing the valves 330 to be human operated. Secondly, since the manifold 300 is replaced after use of the cell culture chamber 200, the valves 330 (which may be the main moving part) are also regularly replaced, thereby reducing the chance of failures due to overuse. Thirdly, the likelihood of errors during setup is also reduced as there is not requirement to locate a flexible tube within a pinch valve. A common failure mode on existing systems is that users will not properly seat the tube to be clamped within the pinch valve, and consequently a complete seal will not be formed.

The manifold 300 may comprise an attachment feature 360 in order to facilitate attachment of the manifold 300 to bioprocessing equipment; for example, the manifold 300 may be attached to a bioreactor apparatus 100, as shown in FIG. 12A. In this example, the manifold 300 comprises two attachment features 360 in the form of protrusions that extend from each side of the housing 310, with each attachment feature 360 having a slot 362.

The fluid conduits 325 may be provided by a matrix 320 as shown in FIGS. 6A to 6D. The matrix 320 comprises interconnected fluid conduits 325, 326 arranged to provide a plurality of first openings 327 and second openings 328 (only some labelled). In this example, the matrix 320 comprises six through-conduits 325-1, 325-2, 325-3, 325-4, 325-5, 325-6, and one cross-conduit 326 that connects to each of the through-conduits 325. The through-conduits 325 are arranged in a substantially parallel configuration with the cross-conduit 326 arranged in a direction substantially perpendicular to the through-conduits 325. The fluid conduits 325, 326 are preferably injection moulded. This allows the matrix 320 to be formed from one common moulding tool, thereby simplifying manufacture of the manifold 300. Each of the fluid conduits 325, 326 is sealable such that fluid flow can be inhibited between one or more of the first openings 327 and one or more of the second openings 328. For example, the fluid conduits 325, 326 may be formed from a thermoplastic material such as CFlex®. In this way, a sealing tool 380 may be used to weld shut one or more of the fluid conduits 325, 326.

A schematic diagram of a sealing tool 380 is shown in FIG. 6B. The sealing tool 380 comprises a number of welding portions 382 (only some labelled) arranged to seal the fluid conduits 320 in a number of positions. For example, there may be welding portions 382a for welding each through-conduit 325 on either or both sides of the intersection with the cross-conduit 326. There may be welding portions 382b for welding the cross conduit 326 between each intersection with the through-conduits 325. In this way, it is possible to manufacture a single matrix 320, which can then be easily configured to define various different fluid path configurations, simply by selectively sealing predetermined fluid conduits 325, 326.

An example of a sealed matrix 320 is shown in FIG. 6C, where dotted lines indicate the sealed portions of the fluid conduits 325, 326. In this example, flow from the first opening 327 of the first through-conduit 325-1 is split such that it exits the second opening 328 of both the first through-conduit 325-1 and the second through-conduit 325-2. Conversely, flow into the first opening 327 of the third through-conduit 325-3 is combined with flow into the first opening 327 of the fourth through-conduit 325-4 to output fluid through the second opening 328 of the fourth through-conduit 325-4. The fifth through-conduit 325-5 and the sixth through-conduit 325-6 are each sealed such that fluid flows directly from their corresponding first opening 327 to the second opening 328. It will be appreciated that this configuration is merely exemplary, and other configurations may be used depending on how the matrix 320 is intended to be used. FIG. 6D shows the configuration of welding portions 382 on the sealing tool 380 that may be activated to form the sealed matrix 320 shown in FIG. 6C. The fluid conduits 325, 326 of the matrix 320 are preferably sealed during the manufacturing of a particular manifold 300 for a particular cell culture chamber 200. Alternatively or additionally, an unsealed matrix 320 may be provided, which may be sealed by an operated before use; in this case, the user may use a hand-operated heat sealing tool.

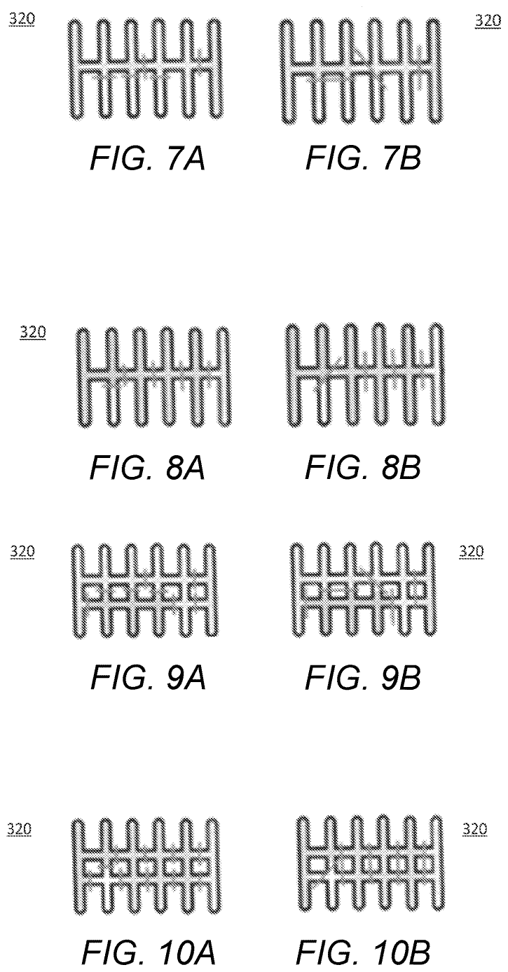

While the sealing tool 380 is one way to seal the fluid conduits 325, 326, other sealing tools such as hand operated sealing tools may be used. As shown in FIGS. 7A and 7B, a longer sealing tool may be used to seal more than one fluid conduit 325, 236 simultaneously, while still providing the same final arrangement of fluid path configurations. Furthermore, while this embodiment is exemplified using heat sealing, alternatively, a sealing tool may be used to mechanically pinch the fluid conduits 325, 326 closed in use, and enabling the fluid paths to be changed during cell processing by adjusting pinch clamp status. The arrangement shown in FIGS. 7A and 7B may be suitable for use with the cell culture chamber 200 described previously. FIGS. 8A and 8B show an alternative arrangement of fluid paths that may be suitable for use with a filtration apparatus.

More than one cross-conduit 326 may be provided to allow further flexibility for different fluid path configurations. FIGS. 9A to 10B show configurations of a tube matrix 320 corresponding to those shown in FIGS. 7A to 8B, but where the tube matrix 320 comprises a first cross-conduit 326-1 and a second cross-conduit 326-2. As particularly shown in FIGS. 9A and 9B, this allows for further fluid paths to be included using the second openings 328 of the second, third, and fourth through-conduits 325.

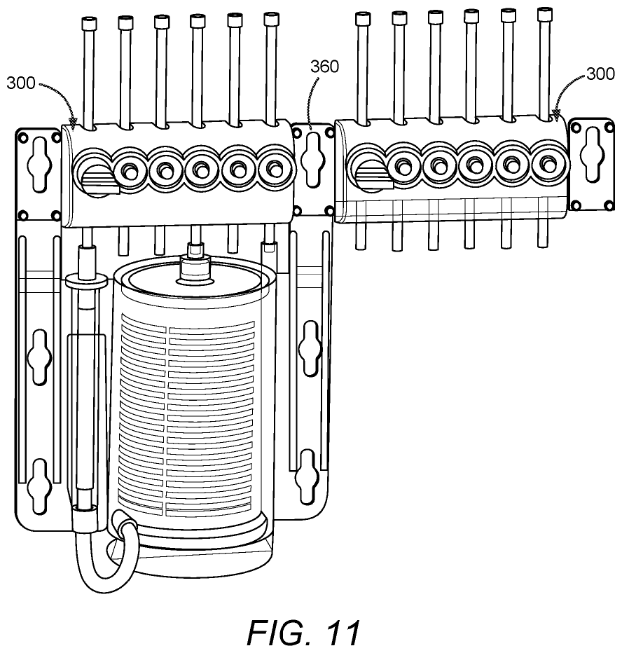

As shown in FIG. 11, more than one manifold 300 may be stacked adjacent to each other. This may reduce the number of manual connections required for adjacent unit operations, since connections need not be formed via an intermediate bag. For example, an output fluid conduit 325 of a first manifold 300 may be attached to an input fluid conduit 325 of a second manifold 300, thereby increasing controllability and configurability of the fluid connections. By allowing manifolds to be stacked adjacent to each other, these connections may be facilitated in a space efficient manner. Adjacent manifolds 300 may be connected together using their corresponding attachment features 360.

Referring now to FIGS. 12A to 12E, an embodiment of the bioreactor apparatus 100 will now be described. The bioreactor apparatus 100 comprises a housing 110 with an external front facing surface 112, and an upper surface 114. The housing 110 has at least one internal cavity 115 for receiving therein a cell culture chamber 200. In this example, the bioreactor apparatus 100 comprises four cavities 115, but any number of cavities 115 may be present. As depicted in FIG. 12A, the leftmost three cavities 115 contain a corresponding cell culture chamber 200, with the rightmost cavity 115 empty. In FIGS. 12D and 12E, the rightmost cavity 115 contains a cell culture chamber 200 and the leftmost three cavities 115 are empty. Each internal cavity 115 has an opening 116 located on an external front-facing surface 112 of the housing 110. The cavity 115 may comprise a push-latch mechanism configured to secure a corresponding cell culture chamber 200. The push-latch mechanism may be spring-loaded so that the cell culture chamber 200 may be readily removed from the bioreactor apparatus 100 by an automated system. The bioreactor apparatus 100 may further comprise at least one movable cover 118 provided on the external front-facing surface 112 of the housing 110, the cover 118 arranged to cover the opening 116 to a corresponding internal cavity 115 when in a closed position. The cover 118 may be configured for automated movement between the closed position and an open position in which the internal cavity 115 can be accessed via the opening 116. As depicted, the leftmost two covers 118 are in the closed position, and the rightmost two covers 118 are in the open position. The cover 118 may be arranged to thermally and/or optically seal the internal cavity 115 when in the closed position.

The housing 110 is configured to incubate the contents of a cell culture chamber 200 disposed within said internal cavity 115. For example, the bioreactor apparatus 100 may comprise a means for supplying heat 190 to a cell culture chamber 200 disposed in an internal cavity 115 of the housing 110. The means for supplying heat 190 may be arranged to be in contact with, or in close proximity to, a cell culture chamber 200 when the cell culture chamber 200 is inserted into the cavity 115. Preferably, the means for supplying heat 190 is arranged to supply heat to the heat exchange portion 224 of the cell culture chamber 200 thereby allowing the medium 250 to be heated before it enters the second housing 230. Preferably, where the bioreactor apparatus 100 comprises a plurality of cavities 115, the housing 110 is configured to incubate individually each cell culture chamber 200 contained within an internal cavity 115 of the housing 110. For example, each cavity 115 may have a corresponding separate means for supplying heat 190. Each of the cavities 115 may be thermally insulated from each other. Advantageously, this allows for separate temperature control for separate cell culture chambers 200 disposed within each cavity 115.

The bioreactor apparatus 100 may further comprise means for agitating 192 a cell culture chamber 200 disposed within an internal cavity 115 of the housing 110. Agitation of the cell culture chamber 200 advantageously allows the cells 251 to be resuspended within the medium 250 in the cell culture chamber 200; this means that samples 255 taken from the cell culture chamber 200 are better representative of the cells 251 growing throughout the cell culture chamber 200. The means for agitating 192 may comprise a rocker, orbital shaker, vibration source, and/or a magnetic stirring means. With a rocker, it may be possible to operate the bioreactor apparatus 100 as a wave bioreactor, where the cell culture chamber 200 is rocked during perfusion. Alternatively, an external automated means may shake the cell culture chamber 200. As a further alternative, fluid may be rapidly pumped and circulated through the cell culture chamber 200 to disturb and resuspend the cells 251.

Preferably, the means for agitating 192 comprises an ultrasound source. In this way, it may be possible to agitate the cell culture chamber 200 without the need to accommodate relative movement of the cell culture chamber 200 in the cavity 115 (as would be required with a rocker or vibration source). This allows the cell culture chamber 200 to be more securely retained in the cavity 115 and may reduce the form factor of the bioreactor apparatus 100. The ultrasound source 192 may comprise an ultrasonic array. In this way, the ultrasound waves may be directed to specific parts of the cell culture chamber 200, such as to selectively resuspend a certain of area of cells 251 for sampling, without disturbing other cells 251 contained in the cell culture chamber 200. Alternatively, or additionally, the ultrasound source 192 may be operated for sedimentation of the cells 251 by setting up standing waves that cause the cells 251 to cluster together and fall to the bottom of the cell culture chamber 200.

The housing 110 is further configured to have mounted to it at least one holding device 150 for supporting a container 160 of a fluid for fluid connection to a cell culture chamber 200 disposed in the internal cavity 115. Specifically, the housing 110 comprises at least one docking port 120 located in the external front-facing surface 112 of the housing 110. An example of a holding device 150 is shown in FIG. 12B, though as shown in FIG. 12A different holding devices 150 may be used for holding different containers 160. For example, there may be separate containers 160 for fresh medium, cytokines, cells, waste or sample extraction, and/or any other suitable container 160. Each holding device 150 may have different dimensions that may correspond to the dimensions of the corresponding container 160.

In the embodiment shown in FIG. 12B, the holding device 150 (e.g. holder for a container 160) comprises a substantially rectangular frame 152. Within the frame 152 is suspended a container 160 (e.g. a “consumable” fluid bag). The container 160 can be attached to the frame 152 via a clip/tag 154, for example, provided on the frame 152. The frame 152 comprises a “leg portion” (or “rod”) 153 that extends down from one side of the frame 152, and then across substantially the width of the frame 152, in parallel to a lower portion 156 of the frame 152 to provide a support for the frame 152. The frame 152 and rod 153 may be formed from a metal material, preferably stainless steel, or a suitably rigid plastic material. A tube clip 158 may be attached to a side of the frame 152 for retaining a portion of tube that is fluidly connected to a container 160, when mounted to the frame 152. The tube clip 158 ensures that at least said portion of the tube is retained in a predetermined position, for example so that it can readily found and/or engaged by a robotic device (or other automated means for manipulating a tube).

In this embodiment of a holding device 150, the rod 153 is configured to be inserted into a docking port 120 on the external front-facing surface 112 of the housing 110. The docking port 120 may comprise an opening to a correspondingly sized elongate slot or hole that extends into the housing 110.

The housing 110 may be further configured to support the lower portion 156 of the holding device 150, such that the frame 150 physically rests on the housing 110. For example, the housing 110 may comprise a groove 125 on an upper surface 114 of the housing 110, the groove 125 configured to contact a support portion 156 of the frame 152 when the rod 153 is inserted into the docking port 120 in the housing 110. Preferably, each docking port 120 has a corresponding groove 125 located above the docking port 120 on an upper surface 114 of the housing 110. In this way, the frame 152 and the container 160 are maintained in a substantially fixed position relative to the housing 110, such as in an upright position. Preferably, the container 160 is supported by the holding device 150 in an elevated position relative to the cavity 115 for receiving the cell culture chamber 200; in this way, a pump is not required to transfer fluid from the container 160 to the cell culture chamber 200.

Each container 160 may be connected to one or more fluid conduits 162, such as one or more tubes 162. As shown in FIGS. 12A and 12B, an upper tube clip 158 may be mounted to the frame 152 to retain the tube 162 connecting to the container 160. A plurality of lower tube clips 128 may be located on the front-facing surface 112 of the housing 110. Each lower tube clip 128 is arranged to retain the tube 162 connected to each container 160 when the corresponding holding device 150 is inserted into the docking port 120 on the housing 110.

The front-facing surface 112 of the housing 110 may comprise one or more fasteners 170 arranged to secure a manifold 300 on the front-facing surface 112 of the housing 110. In this example, each fastener 170 is a bar 170 shaped to fit through a slot 362 in the attachment feature 360 of the manifold 300. Subsequently, the bar 170 may be rotated relative to the slot 362, either by an automated means or by a human operator, thereby pinning the manifold 300 to the housing 110.

The bioreactor apparatus 100 may also comprise a plurality of valving actuators 172. Each valving actuator 172 is arranged to engage with a corresponding valve 330 on the manifold 300 when the cell culture chamber 200 is inserted into the cavity 115 and its corresponding manifold 300 is attached to the front-facing surface 112 of the housing 110, such as by the fasteners 170. Each valving actuator 172 may move between a retracted position, where the valving actuator 172 does not pinch shut a fluid conduit 325 in the valve 330, and an extended position, where the valving actuator 172 extends into the opening 335 on the rear portion 313 of the housing 310 and presses against the fluid conduit 325 in the valve 330 thereby pinching the fluid conduit 325 shut and inhibiting flow of fluid therethrough. The valving actuators 172 may comprise a solenoid mechanism.

The bioreactor apparatus 100 may also comprise a gas port 175. The gas port 175 is configured to supply gas to the cell culture chamber 200 stored within each cavity 115 of the bioreactor apparatus 100. In this example, there is a gas port 175 for each of the cavities 115 thereby facilitating independent gas supply to each of the cell culture chambers 200 within each of the cavities 115. Each gas port 175 may supply a mixture of sterile air and CO2. The gas may be directed from the gas port 175 into the cell culture chamber 200 via a gas connector 176 that is fluidly connected to at least one fluid conduit 325 on the manifold 300. In this example, the gas connector 176 distributes gas from the gas port 175 into the third and fourth fluid conduits 325 in the manifold 300 which subsequently are supplied to the third port 205-3 and fourth port 205-4 of the cell culture chamber 200.

The bioreactor apparatus 100 may be configured to supply gas to each of the cavities 115. For example, where the cell culture chamber 200 comprises a gas permeable membrane 260, oxygen gas may be supplied to each cavity 115 to facilitate growth of cells 251 in the cell culture chamber 200. The covers 118 may be moved to the closed position when gas is supplied to the cavity 118 in order to reduce leakage from the cavity 118. The bioreactor apparatus 100 may comprise at least one gas container (not shown) for supplying fluid to each of the gas ports 175 and/or to each of the cavities 115. Alternatively, an external gas supply (such as within the bioprocessing system) may supply gas to the bioreactor apparatus 100, which may subsequently distribute the gas to each of the gas ports 175 and/or the cavities 115.

The bioreactor apparatus 100 may also comprise a means for pumping 195, for pumping fluid from each of the containers 160 into each cell culture chamber 200. For example, a pump such as a peristaltic pump 195 (preferably a linear peristaltic pump) may be provided inside each cavity 115; when the cell culture chamber 200 is inserted into the cavity 115, the peristaltic pump 195 engages with the flexible tube 272 at the second end 200b of the cell culture chamber 200. In this way, the peristaltic pump 195 may be operated to draw fluid from one or more of the containers 160 and into the cell culture chamber 200. As previously discussed, the pump 195 may be used to operate the cell culture chamber 200 in perfusion, and/or to meter samples 255 into the sampling loop 226 of the cell culture chamber 200.

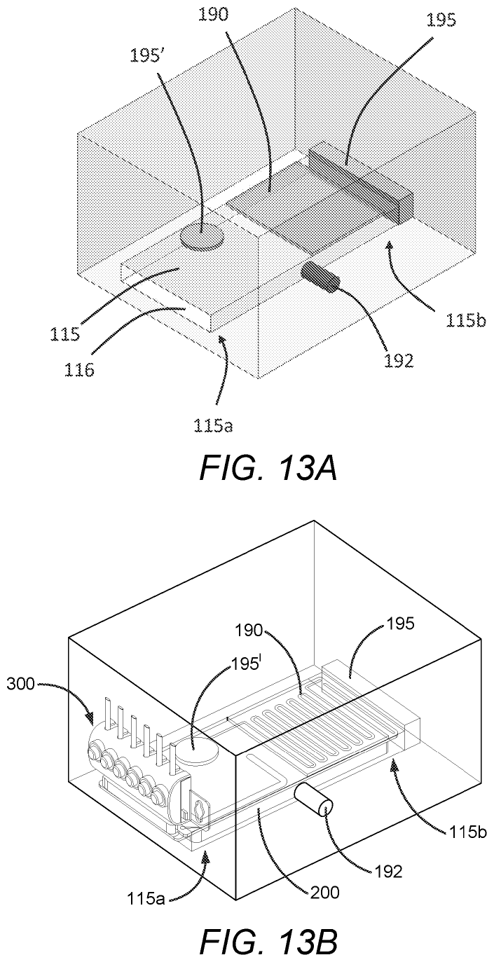

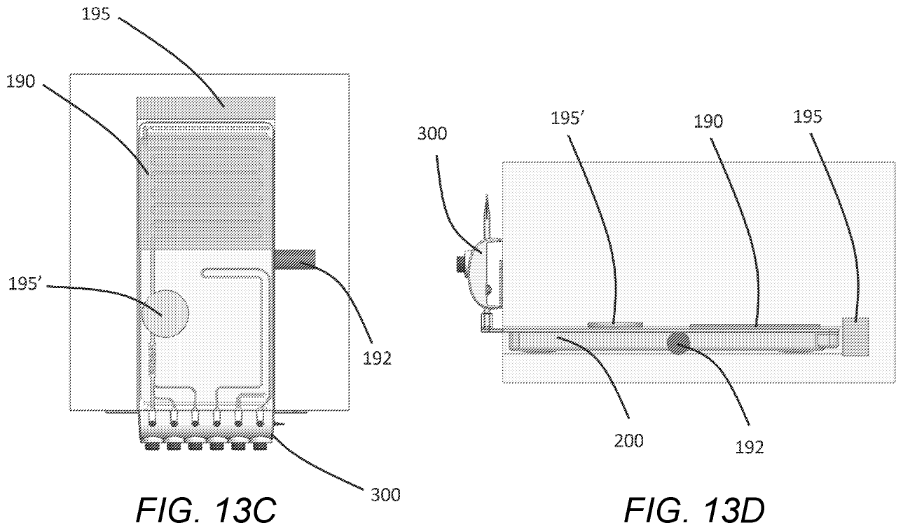

FIGS. 13A to 13D show a schematic internal view of one cavity 115 of the bioreactor apparatus 100 and its surrounding components. The cavity 115 has a first (front) end 115a adjacent to the opening 116 and an opposite second (back) end 115b. FIG. 13A depicts an empty cavity 115, and FIGS. 13B to 13D depict a cavity 115 with a cell culture chamber 200 (with a corresponding manifold 300) inserted. As shown, the means for supplying heat 190 may be arranged adjacent to the cavity 115 so that when the cell culture chamber 200 is inserted, the means for supplying heat 190 is next to the heat exchange portion 224. The peristaltic pump 195 may be arranged at the second end 115b of the cavity 115 such that it is adjacent to the second end 200b of the cell culture chamber 200 when it is inserted into the cavity 115. An alternative or additional means for pumping 195′ may also be provided, which may be a piezo driven valveless pump. The means for agitating 192 such as an ultrasound source 192 may also be located adjacent to the cavity 115. The cavity 115 may comprise one or more LEDs which may be used to sterilise the cavity 115, when the cell culture chamber 200 is removed. For example, the cavity 115 may comprise an LED bank that may be configured to disinfect the cavity 115 after every incubation period. The cover 118 of the cavity 115 may automatically be fully closed during use of the LED bank. Advantageously, this allows safe sterilisation of the cavity 115 without the need for additionally consumables and is solid state.

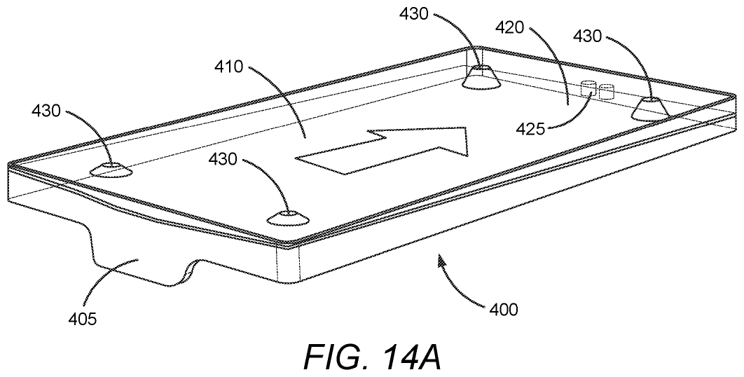

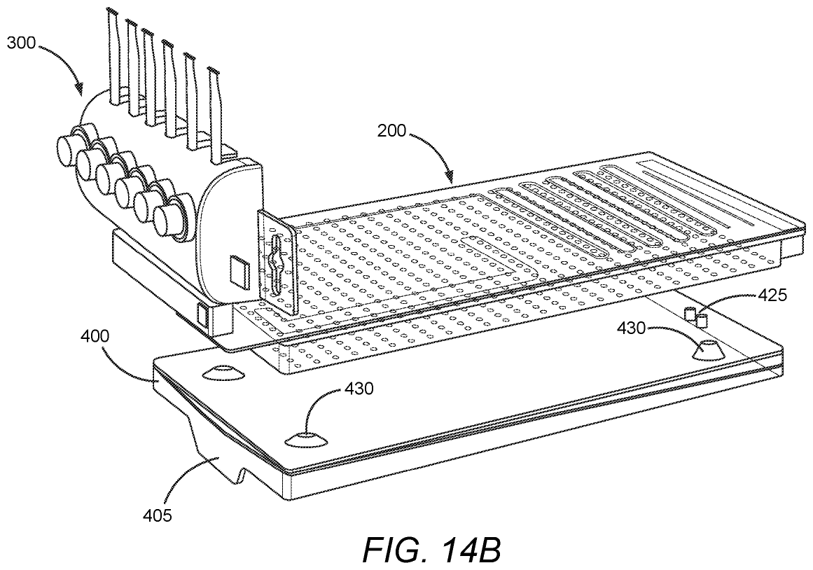

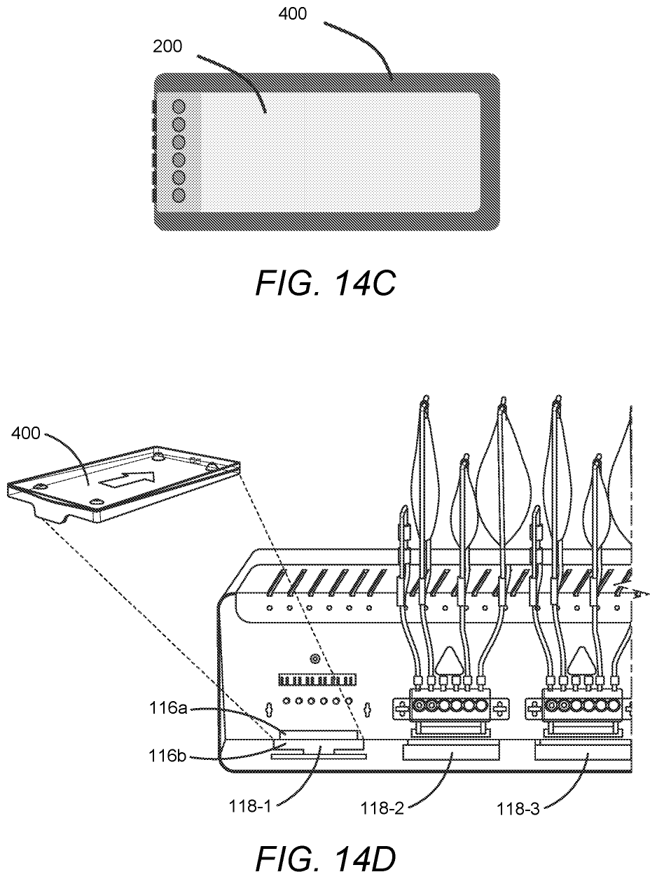

The cavity 115 may also be configured to receive a tray 400, such as the tray 400 or “drip tray” shown in FIGS. 14A to 14C. The tray 400 is configured to be located below the cell culture chamber 200 inside a cavity 115 of the bioreactor apparatus 100. In this way, any leaks from the cell culture chamber 200 may be collected by the tray 400 rather than the bioreactor apparatus 100, which may thereby simplify cleaning of the bioreactor apparatus 100. Since leaks from the cell culture chamber 200 are likely to be rare, the tray 400 may remain in the cavity 115 of the bioreactor apparatus 100 even when multiple cell culture chambers 200 are inserted or removed. In this way, when a leak is detected, the tray 400 may be removed and cleaned without the need to clean the bioreactor apparatus 100.

The tray 400 may be a one-piece injection moulded or vacuum formed construction. The tray 400 is preferably durable, sterilisable and/or reusable. The tray 400 may have a handle 405 so that it may easily by inserted and removed from the cavity 115. The tray 400 may have an angled base 410 to channel fluid towards a collection portion 420 of the tray 400. In this example, the collection portion 420 is at one end of the tray 400. One or more sensors 425 may be arranged to detect the presence of moisture in the collection portion 420. For example, the sensors 425 may be built into the tray 400, and/or may be part of the bioreactor apparatus 100. Where the sensors 425 are part of the tray 400, the tray 400 may have electrical contacts arranged to contact a corresponding pad on the bioreactor apparatus 100 when the tray 400 is inserted into the cavity 115. In this way, data from the sensors 425 may be communicated to the bioreactor apparatus 100, which may be able to alert a user when a leak occurs. The sensors 425 may comprise one or more of: a capacitive sensor, a humidity sensor, an IR LED or other optical sensor, and ultrasonic sensor, a laser level transmitter.

The tray 400 comprises a plurality of supports 430 upon which the expansion chamber 200 is configured to rest when inserted into the cavity 115 of the bioreactor apparatus 100. In this example, four supports 430 are present, but it will be appreciated that any number of supports 430 may be included. The supports 430 are preferably rounded so that the tray 400 may be more easily cleaned. By providing supports 430, gas may flow to the underside of the cell culture chamber 200 such as to reach the gas permeable membrane 260.

As particularly shown in the top-down view in FIG. 14C, the tray 400 is wider and longer than the cell culture chamber 200, to increase the chance that leaks from the cell culture chamber 200 are caught by the tray 400.

As shown in FIG. 14D, the opening 116 of bioreactor apparatus 100 may have a first portion 116a to receive the cell culture chamber 200 and a portion 116b to receive the tray 400. As will now be described in relation to FIG. 14D, the covers 118 corresponding to each cavity 115 may be moved between different positions. For example, the cover 118-1 corresponding to the leftmost (first) cavity 115 is in a fully open position where the tray 400 may be inserted or removed into the second opening portion 116b, such as for cleaning or replacement. The cover 118-2 corresponding to the adjacent (second) cavity 115 is shown in a partially open position, where the cell culture chamber 200 may be inserted or removed into the first opening portion 116a. This position may also allow observation of the cavity 115 such as to observe the cell culture chamber 200 and/or the tray 400. The cover 118-3 corresponding to the adjacent (third) cavity 115 is shown in a mostly closed position, which may be used for incubation of the cell culture chamber 200. Each cover 118 may also move to a fully closed position. The fully closed position may allow the cavity 115 to be UV sterilised such as after an expansion cycle.

The cover 118 may also be moved to the fully open position to enable manual cleaning of the cavity 115 such as once the tray 400 is removed. This exposes both portions 116a, 116b of the opening 116, which provides a gap large enough (e.g. at least 20 mm) for a cleaning tool to be inserted into the cavity 115. The cleaning tool may be a custom cleaning tool which may have a custom piece of foam on a handle. Advantageously, the manual cleaning process is familiar to users, and the planar internal surfaces of the cavity 115 enable easy cleaning. The manual cleaning may be used in combination with the UV sterilisation described previously.

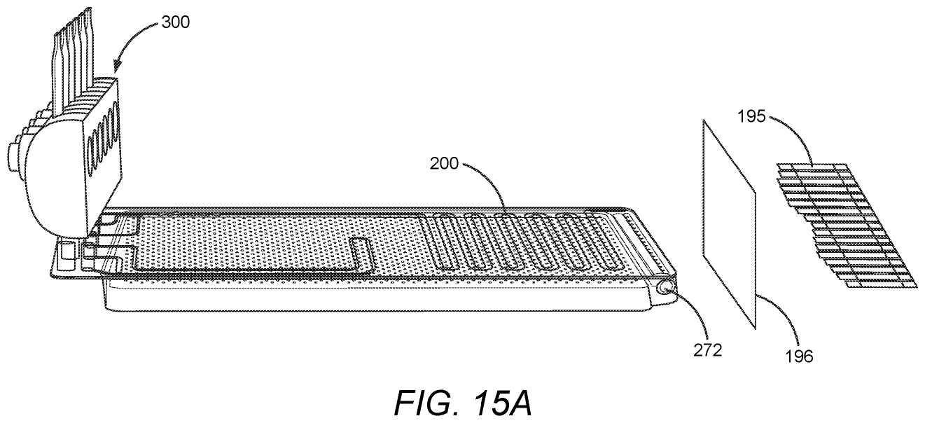

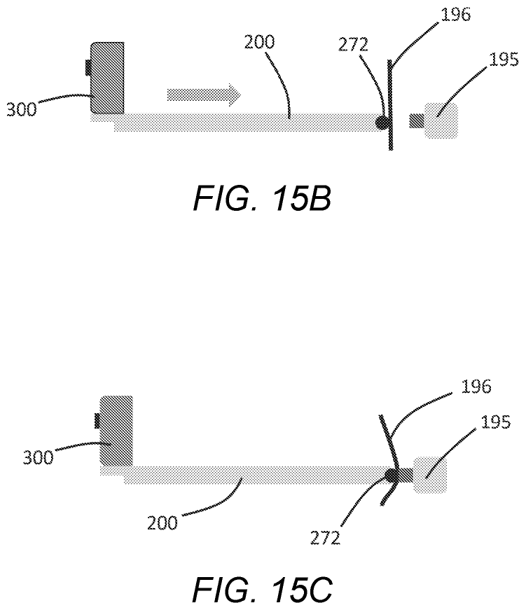

As shown in FIGS. 15A to 15C, the cavity 115 may comprise a barrier such as an elastic barrier 196 between the cell culture chamber 200 and the linear peristaltic pump 195. FIGS. 15A and 15B show the cell culture chamber 200 partially inserted into the cavity 115, and FIG. 15C shows the cell culture chamber 200 fully inserted into the cavity 115. The barrier 196 may be made of an elastic membrane. The barrier 196 may prevent any fluid from reaching the peristaltic pump 195 in case of a leak from the cell culture chamber 200, thereby enabling easy sterilisation of the cavity 115, such as between expansion cycles. When the cell culture chamber 200 is inserted into the cavity 115, the flexible tube 272 of the cell culture chamber 200 is fluidly isolated from the peristaltic pump 195. However, since the barrier 196 is preferably flexible, the peristaltic pump 195 may thereby still apply a peristaltic pumping action to the flexible tube 272 through the barrier 196.

There may be other ways in which leaks from the cell culture chamber 200 may be managed. For example, a disposable plastic film sleeve may be located around each cell culture chamber 200. While this is a simple, low-cost solution that removes the need to clean the bioreactor apparatus 100, it may restrict the gas supply to the gas permeable membrane 260. Furthermore, the disposable plastic film sleeve may be a barrier to heating and/or acoustic streaming. Alternatively, a reusable and cleanable outer housing may be located around each cell culture chamber 200. Again, this is a simple, low-cost solution that removes the need to clean the bioreactor apparatus 100. Furthermore, the outer housing may be provided with moulded geometry to encourage gas flow to the gas-permeable membrane 260. However, the outer housing may provide more of a barrier to heating and acoustic streaming than the film sleeve.

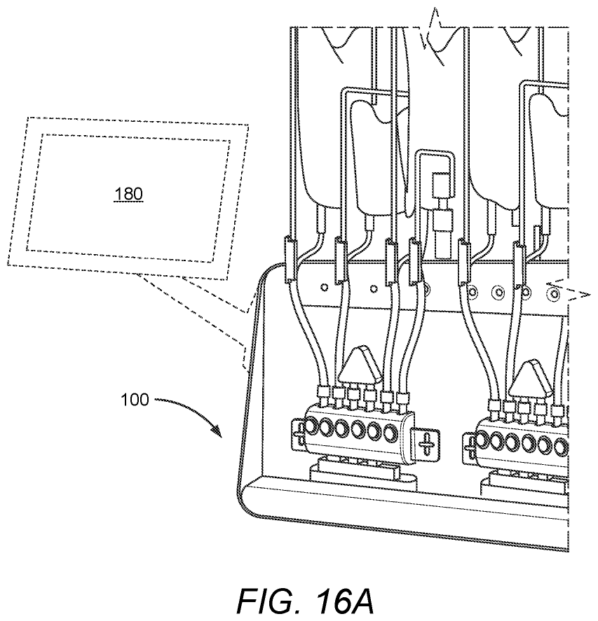

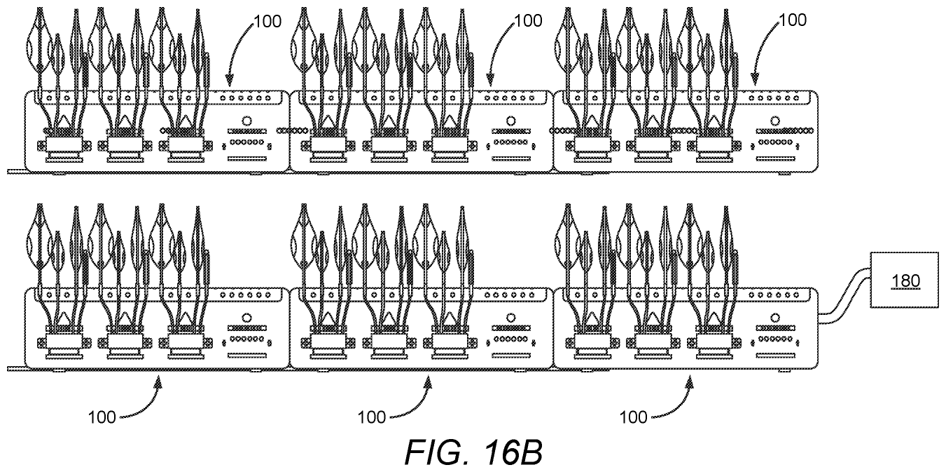

As shown in FIG. 16A, the bioreactor apparatus 100 may be fitted with a control interface 180 such as a tablet device 180. This may allow a user to program a particular sequence of operations to be performed by the bioreactor apparatus 100, or may allow a user to instruct the bioreactor apparatus 100 to perform a particular operation, such as the sampling operation described in relation to FIGS. 3A and 3B. As shown in FIG. 16B, a single control interface 180 may be used to control multiple bioreactor apparatuses 100. Each of the bioreactor apparatuses 100 may be connected together (“daisy-chained”) to facilitate communication with the control interface 180. Each bioreactor apparatus 100 may have tessellation features to enable connection and alignment with an adjacent bioreactor apparatus 100. By connecting together multiple bioreactor apparatuses 100, they may share resources, and the bioprocessing system 1 may be easily scaled through the addition of further bioreactor apparatuses 100. Alternatively, each bioreactor apparatus 100 may be controlled remotely, such as by wireless communication.

The bioreactor apparatus 100 described herein has a number of advantages. The bioreactor apparatus 100 allows the cell culture chambers 200 and/or holding devices 150 to be front-loaded into the bioreactor apparatus 100. This means that an automated system can readily install the cell culture chamber 200 and/or the holding devices 150 into each bioreactor apparatus 100 by simply moving to a predetermined location. Ports such as the gas ports 175 are also provided on the external front facing surface of the bioreactor apparatus 100 thereby facilitating connection to the cell culture chamber 200, such as via the manifold 300. The bioreactor apparatus 100 also includes other features such as the push-latch mechanism that make it particularly suitable for operation by an automated means.

By providing a means for heating adjacent to the cavity 115, fluid may be warmed as it is pumped into the second housing 230 of the cell culture chamber 200. This allows the containers 160 to be stored at room temperature (or optionally refrigerated). This increases the stability and shelf life of the containers 160 and also reduces the energy required to perform a cell therapy process. Furthermore, since each cavity 115 has a small volume and is configured to receive a single cell culture chamber 200, the energy required is reduced, and it is possible to individually control the temperature of separate cell culture chambers 200.

The valves 330 facilitate both manual control, and automated control by the valving actuators 172. This allows a cell therapy process to be performed autonomously, while still allowing a human operator to intervene without needing to remove the cell culture chamber 200 from the bioreactor apparatus 100.

By using an ultrasound source for agitating, the cavity 115 does not need to accommodate relative movement of the cell culture chamber 200 thereby reducing the form factor of the bioreactor apparatus 100. Furthermore, the bioreactor apparatus 100 only requires one pump for each cell culture chamber 200, rather than needing several pumps for engaging with a separate piece of tubing connected to a complex consumable. The bioreactor apparatus 100 also facilitates metered sterile sampling from a cell culture chamber 200 simply by operating a peristaltic pump and supplying air to the cell culture chamber 200.

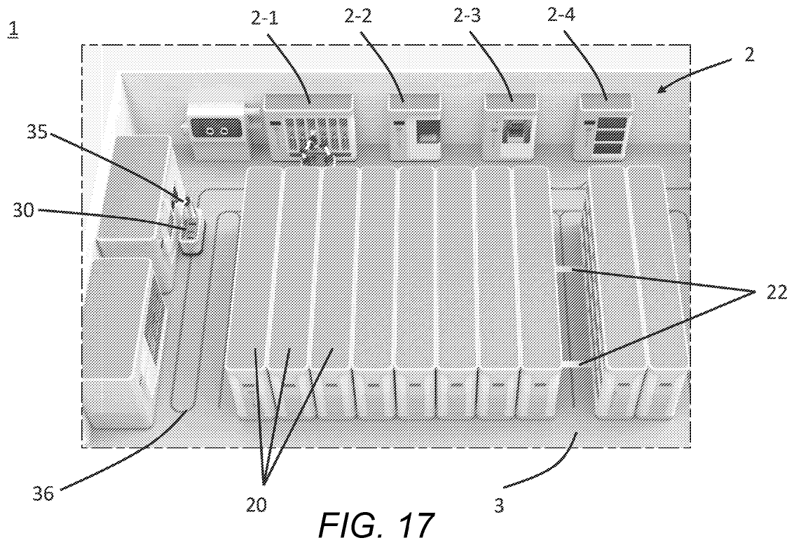

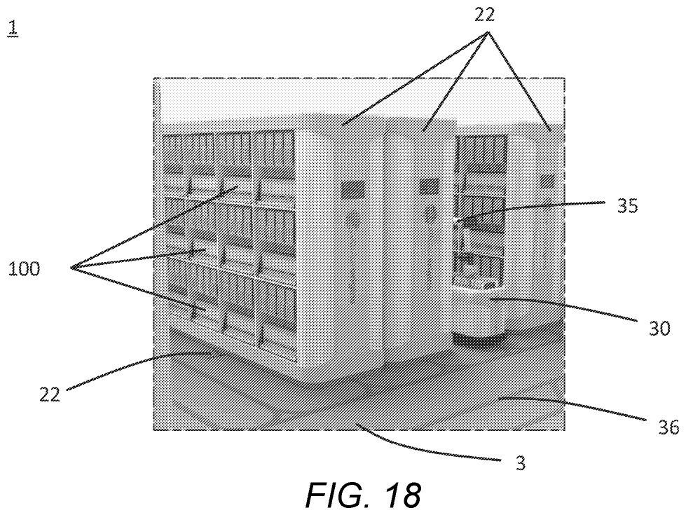

FIGS. 17 and 18 show an embodiment of a bioprocessing system 1. The bioprocessing system 1 may contain a number processing stations 2, such as an enrichment and washing station 2-1, an at-line analytics station 2-2, a fill finish station 2-3, a cryopreservation station 2-4, though other and alternative processing stations may be used. The bioprocessing system 1 is preferably located in a clean room, such as a class C or D clean room. The bioprocessing system 1 includes a plurality of shelving frames (or “shelves”) 20, preferably arranged adjacent to each other. Each shelf 20 is configured to hold a plurality of bioreactor apparatuses 100, where each bioreactor apparatus 100 is configured to receive at least one cell culture chamber 200. While the shelves 20 are shown and described as holding the bioreactor apparatus 100 and cell culture chambers 200 described previously, it will be appreciated that alternative bioreactor apparatuses may be used instead or in addition to those described herein. Each bioreactor apparatus 100 may be configured to carry out steps in a cell therapy process such as activation, transfection, and expansion. For clarity, only some of the shelves 20 and bioreactors 100 have been labelled. The shelves 20 may be mounted upon rails 22 that extend along a length of the bioprocessing system 1. The shelves 20 may be motorized so that the shelves 20 may slide along the rails 22 to move the shelves 20 to different positions in the bioprocessing system 1. The movement of the shelves 20 is preferably automated so that the shelves 20 may be moved to any configuration without the need for a human operator.

The bioprocessing system 1 may also include a robotic device 30 for performing operations in the bioprocessing system 1. The robotic device 30 may be a mobile manipulation unit 30 configured to move freely around a floor 3 of the bioprocessing system 1. In this embodiment, the bioprocessing system 1 comprise a path network 36 upon which the mobile manipulation unit 30 is configured to move. For example, the path network 36 may define paths between the shelves 20 and between the processing stations 2. The mobile manipulation unit 30 may comprise a robotic carrier 35 such as an end effector on a robotic arm.

By providing a bioprocessing system 1 with a plurality of movable shelves 20, the bioprocessing system 1 is very space efficient since the shelves 20 do not need to be permanently spaced apart in order to facilitate access by a robotic device 30 (or a human operator). For example, the bioprocessing system 1 may comprise 1920 bioreactors 100, and may have capacity for about 50,000 cell therapy operations per year. This represents a 10-100 times improvement to space efficiency to existing bioprocessing systems. In addition, by providing the bioprocessing system 1 with a plurality of movable shelves 20, the likelihood of misidentifying a bioreactor apparatus 100 and/or a cell culture chamber 200 may be reduced, since the robotic device 30 needs to actively move the shelves 20 in order to access a particular bioreactor apparatus 100 and/or cell culture chamber 200.

Alternatively, cell culture chambers may be provided on the shelves 20 without being installed in a bioreactor apparatus. When a fluid connection needs to be made to a cell culture chamber, it may be removed from the shelf and transported to a separate location in the bioprocessing system 1 for connection with a bioprocessing consumable (e.g., a media bag, a reagent bag, a waste consumable, or an output consumable for receiving cultured cells). The fluid connection may be made at a bioprocessing apparatus which may be one of the processing stations 2 in the bioprocessing system 1.

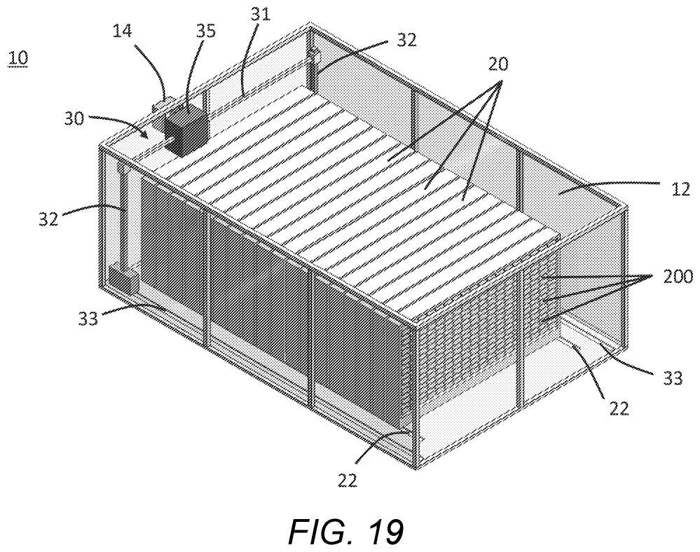

FIG. 19 shows an alternative implementation where the shelves 20 are located in an incubation room 10 which may form part of a bioprocessing system 1. The incubation room 10 is surrounded by walls 12 to provide thermal insulation to the incubation room 10; for example, the walls 12 may comprise a plurality of layers to improve the thermal insulation. By providing an incubation room 10, temperature fluctuations may be reduced due to the larger thermal capacity, and the incubation room 10 may provide a further layer of sterility. While the walls 12 shown in FIG. 19 are transparent, it will be appreciated that the walls 12 may be opaque.

The incubation room 10 includes a plurality of shelves 20 each containing one or more individual cell culture chambers 200, such as those described previously. For clarity, only some of the shelves 20 and the cell culture chambers 200 have been labelled. Although not shown in FIG. 19, the cell culture chambers 200 are preferably located within at least one bioreactor apparatus 100, and the cell culture chambers 200 preferably have a manifold 300 attached thereto. Preferably, the incubation room 10 contains a plurality of shelves 20 arranged adjacent to each other, and preferably each of the shelves 20 contains a plurality of cell culture chambers 200. The shelves 20 may be mounted upon rails 22 that extend along a length of the incubation room 10. The shelves 20 may be motorized so that the shelves 20 may slide along the rails 22 to move the shelves to different positions in the incubation room 10.

The incubation room 10 may also include an airlock 14, which allows objects, such as the cell culture chambers 200, to be moved into and removed from the incubation room 10, while minimizing any change in temperature or other conditions in the incubation room 10. For example, the incubation room 10 may be maintained at a particular temperature in order to provide optimal operating conditions for incubation and cell expansion. Specifically, the airlock 14 may comprise an opening in one of the walls 12 of the incubation room 10, with the opening comprising a pair of doors defining a compartment therebetween into which a cell culture chamber can fit with both doors closed.

The incubation room 10 also includes a robotic device 30 with a robotic carrier 35 for performing operations in the incubation room 10. In this example, the robotic device 30 is a gantry robot 30, though it will be appreciated that other types of robotic device 30 may be used for the same purpose, such as a cable mounted robot. The carrier 35 of the gantry robot 30 is mounted on a horizontal gantry 31, thereby allowing the carrier 35 to move along a lateral axis of the incubation room 10. The horizontal gantry 31 is mounted to at least one vertical gantry 32, thereby allowing the carrier 35 to also move along a vertical axis of the incubation room 10. In this example, two vertical gantries 32 are provided on opposite sides of the incubation room 10 in the lateral direction. The vertical gantries 32 are also mounted to a longitudinal gantry 33, thereby allowing the carrier 35 to also move along a longitudinal axis of the incubation room 10. In this way, the carrier 35 may move along all three axes of the incubation room 10 and thus may be able to perform operations at any position in the incubation room 10. In this example, the longitudinal gantry 33 is a pair of rails 33 that run parallel to the rails 22 upon which the shelves 20 slide, though it will be appreciated that the longitudinal gantry 33 may extend along a direction that is not parallel to the rails 22. It will also be appreciated that the gantry robot 30 may be arranged in other ways; for example, the carrier 35 may be mounted to the vertical gantry 32 rather than the horizontal gantry 31.

The carrier 35 may include a tube welder for forming aseptic connections between different containers. For example, the tube welder may be used to connect each of the containers 160 to the fluid conduits 325 of the manifold 300, and/or to connect the fluid conduits 325 of the manifold 300 to the ports 205 of the cell culture chamber 200. Alternatively, a static or manually operated tube welder may be used to manipulate least one of these connections. While a tube welder is the preferred way to manipulate fluidic connections (e.g., to form aseptic connections), it will be appreciated that the fluid connections may be manipulated in other ways by an automated system. For example, a fluidic connection may be manipulated using reusable or reversible aseptic connectors, connectors with an elastomeric seal, septum and needle connectors, or needle-free connectors. Alternatively, single use aseptic connectors and disconnectors may be used. Alternatively, the fluid connection may be manipulated within a locally aseptic environment such as a sterilisation chamber, which may use an autoclave, laser, steam disinfection or a sterilant.

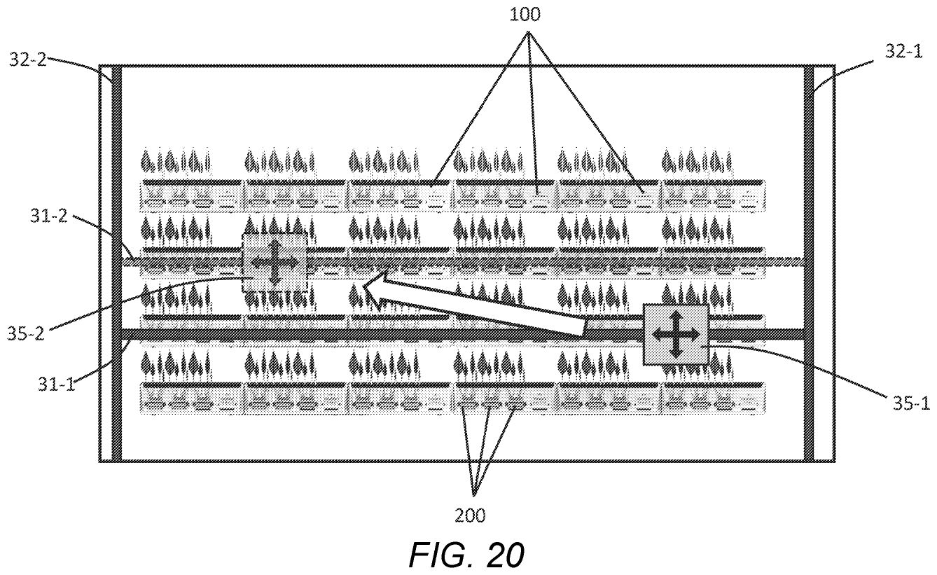

Referring now to FIG. 20, operation of the gantry robot 30 will be described in more detail. FIG. 20 shows a side view of the incubation room 10 viewed along the longitudinal direction, where one shelf 20 containing a plurality of bioreactors 100, each with a plurality of expansion chambers 200. FIG. 20 shows the robotic carrier 35 at a first position 35-1 and at a second position 35-2. To move between the first position 35-1 and the second position 35-2, the horizontal gantry 31 moves from a first position 31-1 to a second position 31-2 that is vertically higher than the first position 31-1. In addition, the robotic carrier 35 moves along the horizontal gantry 31 towards the left. Note that these movements are only exemplary, and the gantry robot 30 may move the robotic carrier 35 to any position in the horizontal and vertical directions in order to access any of the bioreactors 100 within a particular shelf 20. Additionally, the gantry robot 30 may access any of the shelves 20 by moving along the longitudinal direction (not shown in FIG. 20). The shelves 20 may be moved along the rails 22 in order to provide access to the gantry robot 30.

As used herein the term “aseptic” refers to any connection where the contents of the corresponding tubes are not exposed to the surroundings at any stage during the connection (or disconnection) process. This significantly reduces the risk of contamination of either the surroundings or the contents of the tubes. Preferably, the tube welder forms functionally closed, and more preferably fully closed connections between different containers, where a physical barrier is always present between the contents of each containers and the surroundings.

As used herein, the term “tube welder” refers to any device that is configured to join (i.e. weld) a first tube to a second such tube (preferably at their free ends), thereby providing an aseptic (and preferably closed) fluid connection between the tubes. Briefly, a tube welder may comprise a first clamping unit and a second clamping unit. Each clamping unit may comprise a pair of jaws movable between an open position for receiving a flexible tube therebetween, and a closed position for clamping a received tube. The clamping units may be located on a robotic arm. When a tube is clamped, the flexible tube is pinched shut, preferably inhibiting any flow of fluid therethrough. The clamping units may be operated to grip the tubes without clamping them shut; this may enable the tubes to be manipulated to different positions around the bioprocessing system 1 without inhibiting flow of fluid. When a first tube is clamped by the first clamping unit and a second tube is clamped by the second clamping unit, a cutting blade may be heated and moved to intersect a clamped portion of both of the tubes. This cuts each tube into an upstream portion leading to a respective container, and a downstream portion that previously led to a closed end of the tube. Heat from the cutting blade is transferred to the tubes, thereby at least partially melting each flexible tube at the newly formed cut ends. Subsequently, the clamping units are moved so as to locate the upstream portions tubes adjacent to each other. The downstream portions may be discarded. Once the blade is removed, the upstream portions may be pressed into each other, thereby welding the tubes together to form a single tube. The joint may be referred to as a butt-weld. At this stage, the joint between the tubes may remain pinched shut; a pinch release mechanism may be operated to remove the pinched portion, thereby establishing a fluidic path through the joined tubes.

Subsequently, fluid may be pumped through the joined tube in order to perform a transfer of fluid between the respective containers. The peristaltic pump may be operated to pump fluid through the tube. The peristaltic pump may be a rotary peristaltic pump or may be a linear peristaltic pump. In a peristaltic pump, the pump is configured to compress a portion of the flexible tube, and then move the compressed portion along the length of the tube in a pumping direction, thereby forcing fluid through the tube. Once a transfer of fluid is complete, the tube welder may be operated to disconnect the tubes. Alternatively, a separate sealer, such as an RF sealer may be used for this purpose.

While the foregoing is directed to exemplary embodiments of the present invention, it will be understood that such embodiments are described herein purely by way of example, and modifications of detail can be made within the scope of the present invention. Furthermore, one skilled in the art will understand that the present invention may not be limited by the embodiments disclosed herein, or to any details shown in the accompanying figures that are not described in detail herein or defined in the claims. Indeed, such superfluous features may be removed from the figures without prejudice to the present invention.

Moreover, other and further embodiments of the invention will be apparent to those skilled in the art from consideration of the specification and may be devised without departing from the basic scope thereof, which is determined by the claims that follow.

1. A bioprocessing system, comprising:
a plurality of shelving frames, each shelving frame configured to hold a plurality of cell culture chambers; and
an automated system configured to:
move a cell culture chamber around the bioprocessing system; and
manipulate a fluid connection between a cell culture chamber and a bioprocessing consumable;
wherein at least one of the shelving frames is movable relative to at least one other of the shelving frames whereby to facilitate or inhibit access to a cell culture chamber held on one of the shelving frames.

2. The bioprocessing system of claim 1, wherein each shelving frame is configured to hold a plurality of bioreactor apparatus, each bioreactor apparatus being configured to receive at least one cell culture chamber.

3. The bioprocessing system of claim 2, wherein the bioprocessing consumable is provided by a bioreactor apparatus held on one of the shelving frames.

4. The bioprocessing system of claim 1, wherein the automated system is configured to manipulate a fluid connection between the cell culture chamber and the bioprocessing consumable at a bioprocessing apparatus that is located in the bioprocessing system separately to the plurality of shelving frames.

5. The bioprocessing system of claim 1, wherein the shelving frames are mounted on a track system along which each frame can be moved within the enclosed space.

6. The bioprocessing system of claim 1, wherein the automated system comprises:
a gantry comprising a horizontal member mounted slidably to at least one vertical member and configured such that the horizontal member can be moved along the vertical member; and
at least one robotic device mounted slidably to the horizontal member and configured such that the robotic device can move along the horizontal member.

7. The bioprocessing system of claim 1, wherein the automated system comprises a robotic device configured to move across a floor of the bioprocessing system whereby to access each of the cell culture chambers.

8. The bioprocessing system of claim 1, wherein a robotic device of the automated system is configured to releasably engage with a cell culture chamber whereby to move said cell culture chamber.

9. The bioprocessing system of claim 1, wherein a robotic device of the automated system is configured to manipulate said fluid connection.

10. The bioprocessing system of claim 8, wherein a single robotic device is configured both to move a cell culture chamber and to manipulate said fluid connection.

11. The bioprocessing system of claim 1, wherein the plurality of shelving frames are disposed within an enclosed space having an opening through which a cell culture chamber can be passed, the opening comprising a pair of doors defining a compartment therebetween into which the cell culture chamber can fit with both doors closed.

12. The bioprocessing system of claim 11, wherein the enclosed space is substantially thermally sealed thereby to inhibit the transfer of heat between the enclosed space and its surroundings.

13. The bioprocessing system of claim 1, wherein at least one cell culture chamber in each shelving frame has an individual machine-readable identifier, for example a QR code.

14.-36. (canceled)

37. The bioprocessing system of claim 9, wherein a single robotic device is configured both to move a cell culture chamber and to manipulate said fluid connection.
